(19) United States
(12) Patent Application Publication
(10) Pub. No.: US 2016/0317531 A1
(43) Pub. Date: Nov. 3, 2016

(54) RIBONUCLEOTIDE REDUCTASE INHIBITORS SENSITIZE TUMOR CELLS TO DNA DAMAGING AGENTS

(71) Applicant: THE GENERAL HOSPITAL CORPORATION, Boston, MA (US)

(72) Inventor: Bakhos A Tannous, Lynnfield, MA (US)

(21) Appl. No.: 14/900,286

(22) PCT Filed: Jun. 19, 2014
(86) PCT No.: PCT/US14/43119

§ 371 Date: Dec. 21, 2015

Related U.S. Application Data
(60) Provisional application No. 61/837,972, filed on Jun. 21, 2013.

(51) Int. Cl. A61K 31/495; A61K 31/17

(52) U.S. Cl. CPC A61K 31/495; A61K 31/17

(57) ABSTRACT

The present invention relates to methods for treatment of tumors comprising administering to a subject in need thereof a DNA damaging agent and a ribonucleotide reductase inhibitor. The ribonucleotide reductase inhibitor can sensitize tumor cells to the DNA damaging agent, thus permitting greater treatment efficacy than the DNA damaging agent alone. The methods described herein are generally useful for treating any tumor that can benefit from this combination therapy. In particular, the methods described herein are useful for treating tumors that are resistant or have the propensity to develop resistance to certain DNA damaging agents. Further provided in the prevent invention are compositions comprising a DNA damaging agent and a ribonucleotide reductase inhibitor.

### CROSS-REFERENCE TO RELATED APPLICATION

This application claims benefit under 35 U.S.C. §119(e) of U.S. Provisional Application No. 61/837,972 filed Jun. 21, 2013, the contents of which are incorporated herein by reference in its entirety.

### TECHNICAL FIELD

The present invention relates to compositions and methods for cancer treatment and cancer cell sensitization.

### BACKGROUND

Gliomas account for about 60% of all primary central nervous system tumors in the United States. Glioblastoma (GBM or grade IV glioma), which comprises 51.2% of all gliomas, is the most malignant form. Over the last two decades, the major breakthrough in the treatment for GBM has been the addition of the DNA alkylating agent temozolomide (TMZ) to the standard of care including surgery and radiation, yielding an increase in the median survival from 12.1 months to 14.6 months (Stupp, R., et al., The New England journal of medicine 2005, 352, 987-996). An updated survival data analysis from a randomized phase III study revealed an increase in the overall 2 years survival from 10.9% to 27.2% in patients receiving the combined therapy (Stupp, R., et al., The lancet oncology 2009, 10, 459-466). Despite this success, 90% of patients receiving both TMZ and radiation die after 5 years, a colossal failure that has partially attributed to drug resistance.

Drug resistance can generally be categorized as either acquired or intrinsic which, on a molecular level, share several common foundations (Goldie, J. H. Cancer metastasis reviews 2001, 20, 63-68). One of the major predictors of the response of GBM to TMZ is the intrinsic MGMT (06-methylguanine methyl transferase) promoter methylation status (Stupp, R., et al., The lancet oncology 2009, 10, 459-466). TMZ induces methylation of guanine at O6 position, a change that causes a futile cycle of attempted DNA repair, and results in cell apoptosis. MGMT removes the DNA adduct caused by the alkylating agent, resulting in resistance to TMZ therapy. Thus, patients whose tumors have transcriptional silencing of the MGMT gene, mediated by promoter methylation (which occurs in approximately half of GBM tumors (Hau, P., Stupp, R. & Hegi, M. E., Disease markers 2007, 23, 97-104)), are more likely to benefit from the addition of TMZ to their treatment regimen (Stupp, R., et al., The lancet oncology 2009, 10, 459-466; Hegi, M. E., et al., The New England journal of medicine 2005, 352, 997-1003). However, all glioblastomas recur to a tumor lesion with acquired resistance to TMZ, leading to patient death.

Thus, therapies that could overcome both intrinsic and acquired TMZ resistance could greatly increase patient survival.

### SUMMARY

The inventors have identified that a ribonucleotide reductase inhibitor can sensitize tumor cells to a DNA damaging agent to which tumor cells have developed resistance. Accordingly, provided herein are compositions and methods for cancer treatment that take advantage of a sensitizing agent (e.g., a ribonucleotide reductase inhibitor) to enhance the responsiveness of tumor cells to a DNA damaging agent.

In one aspect, provided herein is a method of treating a tumor in a subject, the method comprising administering to the subject a therapeutically effective amount of a DNA damaging agent and a ribonucleotide reductase inhibitor.

In some embodiments, the ribonucleotide reductase inhibitor is selected from a group consisting of hydroxyurea (HU), motexafin gadolinium, fludarabine, cladribine, gemcitabine, tezacitabine, triapine, gallium maltolate, gallium nitrate, and a combination thereof.

In some embodiments, the ribonucleotide reductase inhibitor is hydroxyurea, fludarabine, gemcitabine, or a combination thereof

In some embodiments, the DNA damaging agent is not a ribonucleotide reductase inhibitor.

In some embodiments, the DNA damaging agent is not radiation.

In some embodiments, the DNA damaging agent is a chemotherapeutic agent or a PARP inhibitor.

In some embodiments, the chemotherapeutic agent is selected from a group consisting of temozolomide (TMZ), bendamustine, irinotecan, capecitabine, topotecan, cisplatin, oxaliplatin, carboplatin, nedaplatin, satraplatin, triplatin tetranitrate, camptothecin, cytarabine, fluorouracil, cyclophosphamide, etoposide phosphate, teniposide, doxorubicin, daunoaibicin, pemetrexed, mitomycin C, chlorambucil, and melphalan.

In some embodiments, the chemotherapeutic agent is temozolomide (TMZ).

In some embodiments, the ratio of the DNA damaging agent to the ribonucleotide reductase inhibitor is sufficient to sensitize tumor cells to the DNA damaging agent.

In some embodiments, the tumor is selected from a group consisting of central nervous system (CNS) neoplasm, melanoma, recurrent adult acute lymphoblastic leukemia, recurrent childhood acute lymphoblastic leukemia, Ewing sarcoma, unspecified adult solid tumor, unspecified childhood solid tumor, hepatocellular carcinoma, pancreatic neuroendocrine tumor (e.g., gastrinoma, glucagonoma, insulinoma, islet cell carcinoma, pancreatic polypeptide tumor, recurrent islet cell carcinoma, or somatostatinoma), lung cancer, colorectal cancer, rectal cancer, breast cancer, ovarian cancer, rhabdomyosarcoma, acute myelogenous leukemia, and myelodysplastic syndrome.

In some embodiments, the CNS neoplasm is selected from a group consisting of glioma, glioblastoma, oligodendroglioma, astrocytoma, medulloblastoma, oligoastrocytoma, gliosarcoma, recurrent adult brain tumor, B-cell lymphoma originating in the CNS, childhood high-grade cerebellar astrocytoma, childhood high-grade cerebral astrocytoma, childhood spinal cord neoplasm, childhood brain stem glioma, childhood cerebral astrocytoma, peripheral primitive neuroectodermal tumor, recurrent childhood medulloblastoma, recurrent childhood supratentorial primitive neuroectodermal tumor, and recurrent childhood pineoblastoma.

In some embodiments, the tumor is glioblastoma or melanoma.

In some embodiments, the glioblastoma is recurrent glioblastoma.

In some embodiments, the tumor is resistant to the DNA damaging agent.

In some embodiments, the ribonucleotide reductase inhibitor is administered before the administration of the DNA damaging agent.

In some embodiments, the ribonucleotide reductase inhibitor is administered simultaneously with the administration of the DNA damaging agent.

In some embodiments, the ribonucleotide reductase inhibitor is administered after the administration of the DNA damaging agent.

In some embodiments, the method further comprises providing the subject at least one other anti-cancer treatment.

In some embodiments, the one other anti-cancer treatment is radiation therapy.

In some embodiments, the subject is a mammal.

In some embodiments, the subject is a human.

In another aspect, the invention relates to the use of a ribonucleotide reductase inhibitor in combination with a DNA damaging agent for the preparation of a medicament for the treatment of tumor, wherein the DNA damaging agent is not a ribonucleotide reductase inhibitor.

In some embodiments, the ribonucleotide reductase inhibitor is selected from a group consisting of hydroxyurea, motexafin gadolinium, fludarabine, cladribine, gemcitabine, tezacitabine, triapine, gallium maltolate, and gallium nitrate, and a combination thereof.

In some embodiments, the DNA damaging agent is a chemotherapeutic agent or a PARP inhibitor.

In some embodiments, the chemotherapeutic agent is selected from a group consisting of temozolomide (TMZ), bendamustine, irinotecan, capecitabine, topotecan, cisplatin, oxaliplatin, carboplatin, nedaplatin, satraplatin, triplatin tetranitrate, camptothecin, cytarabine, fluorouracil, cyclophosphamide, etoposide phosphate, teniposide, doxorubicin, daunoaibicin. pemetrexed, mitomycin C, chlorambucil, and melphalan.

In some embodiments, the chemotherapeutic agent is temozolomide (TMZ).

In yet another aspect, provided herein is a pharmaceutical composition comprising an effective amount of a DNA damaging agent and a ribonucleotide reductase inhibitor.

In some embodiments, the DNA damaging agent is not a ribonucleotide reductase inhibitor.

In some embodiments, the DNA damaging agent and the ribonucleotide reductase inhibitor is in a ratio sufficient for the ribonucleotide reductase inhibitor to sensitize tumor cells to the DNA damaging agent.

In some embodiments, the ribonucleotide reductase inhibitor is selected from a group consisting of hydroxyurea, motexafin gadolinium, fludarabine, cladribine, gemcitabine, tezacitabine, triapine, gallium maltolate, gallium nitrate, and a combination thereof.

In some embodiments, the ribonucleotide reductase inhibitor is hydroxyurea, fludarabine, gemcitabine, or a combination thereof

In some embodiments, the DNA damaging agent is a chemotherapeutic agent or a PARP inhibitor.

In some embodiments, the chemotherapeutic agent is selected from a group consisting of temozolomide (TMZ), bendamustine, irinotecan, capecitabine, topotecan, cisplatin, oxaliplatin, carboplatin, nedaplatin, satraplatin, triplatin tetranitrate, camptothecin, cytarabine, fluorouracil, cyclophosphamide, etoposide phosphate, teniposide, doxorubicin, daunoaibicin. pemetrexed, mitomycin C, chlorambucil, and melphalan.

In some embodiments, the chemotherapeutic agent is temozolomide (TMZ).

### BRIEF DESCRIPTION OF THE DRAWINGS

FIGS. 1A-1B show that HU sensitizes glioma cells to TMZ in vitro.

FIG. 1A is a set of graphs showing the effects of TMZ on tumor cells. U87, SNZ308, HS683 parental cells (P) and resistant clones (Res) were treated with different amounts of TMZ. After four days aliquots of conditioned medium were assayed for Gluc activity using 50 μl 2 μg/ml coelenterazine.

FIG. 1B is a set of graphs showing the effects of TMZ+HU on tumor cells. U87 and resistant clones R1/R2 were treated with TMZ and/or 100 μM HU. Cell viability was assessed by the Gluc assay.

FIGS. 2A-2B are graphs showing that HU sensitizes glioma cells to TMZ in vivo. U87 cells (20,000 cells/mouse) expressing Fluc were implanted in the brain of nude mice. One week later, mice were divided in four groups (n=10-20/group) and treated with 1). DMSO vehicle as control, 2). 50 mg/kg HU, 3). 1 mg/kg TMZ or 4). 1 mg/kg TMZ+50 mg/kg HU. In FIG. 2A, tumor growth was monitored over time by Fluc imaging. X-axis, days after treatment starts. In FIG. 2B, survival was recorded to generate the Kaplan-Meier plot. X-axis, days after treatment starts.

FIGS. 2C-2D are graphs showing that HU sensitizes glioma cells to TMZ in vivo. U87 cells (20,000 cells/mouse) expressing Fluc were implanted in the brain of nude mice. One week later, mice were divided in four groups (n=10-20/group) and treated with 1). DMSO vehicle as control, 2). 50 mg/kg HU, 3). 5 mg/kg TMZ or 4). 5 mg/kg TMZ+50 mg/kg HU. In FIG. 2C, tumor growth was monitored over time by Fluc imaging. X-axis, days after treatment starts. In FIG. 2D, survival was recorded to generate the Kaplan-Meier plot. X-axis, days after treatment starts.

FIGS. 2E-2F are graphs showing that HU sensitizes glioma cells to TMZ in vivo. U87R1 cells (20,000 cells/mouse) expressing Fluc were implanted in the brain of nude mice. One week later, mice were divided in four groups (n=10-20/group) and treated with 1). DMSO vehicle as control, 2). 50 mg/kg HU, 3). 5 mg/kg TMZ or 4). 5 mg/kg TMZ+50 mg/kg HU. In FIG. 2E, tumor growth was monitored over time by Fluc imaging. X-axis, days after treatment starts. In FIG. 2F, survival was recorded to generate the Kaplan-Meier plot. X-axis, days after treatment starts.

FIG. 2G is a graph showing that HU sensitizes glioma cells to TMZ in vivo. 200,000 U87R1 cells expressing Fluc were implanted in the brain of nude mice. Two weeks later, mice were divided in two groups (n=10/group) and treated with either TMZ or TMZ+HU. Survival was recorded.

FIG. 2H is a set of images and a graph showing enhanced survival rate for mice treated with radiation (R)+TMZ+HU. U87R1 cells expressing Fluc were implanted in the brain of nude mice. One week later, mice were divided in four groups (n=10-20/group) and treated with either DMSO vehicle, radiation (R; 3 Gy), HU, TMZ or R+TMZ+HU. Tumor growth was monitored over time by Fluc imaging and survival was recorded to generate the Kaplan-Meier plot.

FIGS. 3A-3F show the effect of HU+TMZ on GBM spheres with different MGMT status.

FIG. 3A is an image of glioma spheres transduced with a lentivirus vector expressing both Gluc and GFP as observed by GFP levels.

FIG. 3B shows images of glioma spheres stained for Nestin and CD133, markers for stem cells and nuclei (Dapi).

FIG. 3C is a graph showing linearity of Gluc secretion with respect to cell number. Different numbers of GBM cells expressing Gluc were plated in a 96-well plate. After 24 hours, 20 μl aliquots were transferred into a new plate and Gluc activity was monitored by addition of 20 μM coelenterazine using a luminometer.

FIG. 3D is a graph showing that the release of Gluc to the conditioned medium is linearly related with respect to cell number and cell growth over time. GBM cells expressing Gluc were plated in 96-well plate and Gluc secretion was monitored overtime as in FIG. 3A.

FIG. 3E is a set of images of GBM neural spheres from newly diagnosed GBM tumors with methylated and unmethylated MGMT promoter and spheres from recurrent GBM plated in a 96-well plate and treated with HU and/or TMZ.

FIG. 3F is a set of graphs analyzing spheres recovery and secondary sphere formation with the different treatment strategies.

FIGS. 4A-4H show that HU sensitizes primary GBM8 to TMZ in vivo.

FIG. 4A is an image showing that 10⁵ GBM8 cells expressing Fluc and mCherry were stereotactically injected into the left midstriatum of nude mice brain.

FIG. 4B is a set of images showing that tumor growth was monitored by Fluc imaging.

FIG. 4C are microscopy images. At the last imaging point, mice were sacrificed and brains were sectioned and analyzed for mCherry using microscopy. GBM8 infiltrated the brain from the left to the right side via the corpus callosum (cc). White arrow, injection site. Bar, 500 μm.

FIG. 4D is a set of images showing the in vivo effects of TMZ+HU treatments on mice. Mice-bearing GBM8-Fluc-mCherry tumors were treated with TMZ or vehicle for two weeks, then left off TMZ for three weeks. TMZ-treated group was then divided into four subgroups which received vehicle, HU, TMZ, or HU+TMZ.

FIG. 4E is a graph monitoring tumor growth.

FIG. 4F is a graph analyzing survival based on FIG. 4E.

FIG. 4G shows images and graphs of mice-bearing MGG23-Fluc tumors with unmethylated MGMT promoter treated with either vehicle (control), TMZ, HU or HU+TMZ. Tumor growth was monitored by Fluc imaging once/week.

FIG. 4H shows microscopy images analyzing bone marrow smear of mice treated with DMSO or HU+TMZ for four consecutive days.

FIG. 5A is a plot showing that glioma cells are sensitized to TMZ by ribonucleotide reductase inhibition. 3×10³ TMZ-resistant primary glioma rpGBMa cells expressing Gluc were treated with 0-100 of ribonucleotide reductase (RNR) inhibitor Fludarabine or Gemcitabine, in the presence or absence of 100 μM TMZ. After four days aliquots of conditioned medium were assayed for Gluc activity using 50 μl 2 μg/ml coelenterazine. Data presented as the mean Gluc RLU/mL +/−SD (n=8; **p<0.01 vs. no TMZ treatment by two-way ANOVA).

FIG. 5B is a plot showing that glioma cells are sensitized to TMZ by ribonucleotide reductase inhibition. U87 and resistant clones R1 cells were infected with lentiviral vector expressing scrambled shRNA (shScrama) or shRNA against RNR M2 subunit (shRRM2) and treated with 0-100 μM TMZ. Cell viability was monitored with alamar blue assay. Data presented as the mean Gluc RLU/mL +/−SD (n=8; **p<0.01 vs. shScram vs. shRRM2 expressing cells by two-way ANOVA).

FIG. 5C is a plot showing that glioma cells are sensitized to TMZ by ribonucleotide reductase inhibition. TMZ-sensitive MGG6 and TMZ-resistant MGG23 primary glioma cultures were infected with shScrama or shRRM2 and treated with 0-100 μM TMZ. Cell viability was monitored with alamar blue assay. Data presented as the mean Gluc RLU/mL +/−SD (n=8; **p<0.01 vs. shScram vs. shRRM2 expressing cells by two-way ANOVA).

FIG. 5D is a plot showing that RNR inhibition sensitizes glioma cells to TMZ in vivo. The left forebrains of mice were implanted with 2×10⁴ U87 cells expressing Fluc and mCherry and infected with shScrama or shRRM. Each group of mice was divided into 2 subgroups which received an i.p. injection (3 times per week over 3 weeks) of either DMSO or TMZ (30 mg/kg body weight). Tumor-associated photon counts were quantified using an IVIS imaging system software. Data presented as the mean of total flux of Fluc (photons/sec) +/−SD (n=6; **p<0.01 vs. shScram +DMSO1; ##p<0.01 vs. shScram or DMSO alone by ANOVA and Tukey's post-hoc test).

FIG. 5E is a set of bioluminescence images showing that RNR inhibition sensitizes glioma cells to TMZ in vivo. In vivo Fluc bioluminescence imaging was performed once/week using the Xenogen IVIS 200 Imaging System to monitor tumor growth. Representative images at 0, 10, and 17 days post-TMZ treatment are shown. Pseudo-color represents radiance intensity of the tumors (photons/sec/cm²/surface radiance).

FIG. 6 shows an image and a graph showing that glioma cells are sensitized to TMZ by γ-secretase inhibition. 293T cells were infected with lenti-RBP-Jk-Fluc vector and treated with 50 μM HU or γ-secretase inhibitor DAPT for 3 hours. Cell lysates was imaged and analyzed for Fluc activity with the Xenogen IVIS 200 Imaging System. **p<0.01 vs. DMSO (n=8 for DMSO, n=5 for HU or DAPT treatment).

FIG. 7 is a graph showing that hydroxyurea sensitizes melanoma cells to TMZ.

### DETAILED DESCRIPTION

The invention is based, in part, on the discovery that a ribonucleotide reductase inhibitor sensitizes tumor cells to a DNA damaging agent to which the tumor cells have developed resistance. By sensitizing the tumor cells, the tumor cells become more responsive to the DNA damaging agent, thereby resulting in greater treatment efficacy. In a non-limiting example, the inventors identified hydroxyurea (HU), an FDA-approved drug, to sensitize TMZ-resistant glioblastoma (GBM) cells to TMZ, both in culture and in primary GBM in vivo intracranial models. Thus, embodiments of the invention provide compositions and methods for treatment of tumors, e.g., a tumor that is resistant to a DNA damaging agent.

Accordingly, one aspect of the invention relates to a method of treating tumor in a subject, the method comprising administering to the subject a therapeutically effective amount of a DNA damaging agent and a ribonucleotide reductase inhibitor. In some embodiments, the DNA damaging agent is not a ribonucleotide reductase inhibitor. In some embodiments, the DNA damaging agent is not radiation.

In some embodiments, the ribonucleotide reductase inhibitor is administered before the administration of the DNA damaging agent. For example, the ribonucleotide reductase inhibitor can be administered 5 minutes or less, 10 minutes or less, 20 minutes or less, 30 minutes or less, 40 minutes or less, 50 minutes or less, 1 hour or less, 2 hours or less, 6 hours or less, 12 hours or less, 18 hours or less, 1 day or less, 2 days or less, 3 days or less, 10 days or less, before the administration of the DNA damaging agent.

In some embodiments, the ribonucleotide reductase inhibitor is administered simultaneously with the administration of the DNA damaging agent. In these embodiments, the ribonucleotide reductase inhibitor and the DNA damaging agent can be administered in the same composition (e.g., a drug formulation comprising the ribonucleotide reductase inhibitor and the DNA damaging agent) or in different compositions.

In some embodiments, the ribonucleotide reductase inhibitor is administered after the administration of the DNA damaging agent. For example, the ribonucleotide reductase inhibitor can be administered 5 minutes or less, 10 minutes or less, 20 minutes or less, 30 minutes or less, 40 minutes or less, 50 minutes or less, 1 hour or less, 2 hours or less, 6 hours or less, 12 hours or less, 18 hours or less, 1 day or less, 2 days or less, 3 days or less, 10 days or less, after the administration of the DNA damaging agent.

As used herein, the term “DNA damaging agent” refers to any agent or treatment that directly or indirectly damages DNA.

In some embodiments, the DNA damaging agent is a chemotherapeutic agent. As used herein, “chemotherapeutic agent” refers to an agent that inhibits or prevents the viability and/or function of cells, and/or causes destruction of cells (cell death), and/or exerts anti-neoplastic/anti-proliferative effects, for example, prevents directly or indirectly the development, maturation or spread of neoplastic tumor cells. The term also includes such agents that cause a cytostatic effect only and not a mere cytotoxic effect. A chemotherapeutic agent can be an organic molecule, an inorganic molecule, or a biological molecule (e.g., nucleic acids, proteins, lipids, peptides, or polysaccharides). A chemotherapeutic agent can be synthetic or naturally occurring. Chemotherapeutic agent does not include radiation. The chemotherapeutic agent is a DNA damaging agent.

A chemotherapeutic agent may be, but is not limited to, those selected from among the following groups of compounds: platinum compounds, cytotoxic antibiotics, antimetabolites, anti-mitotic agents, alkylating agents, arsenic compounds, DNA topoisomerase inhibitors, taxanes, nucleoside analogues, plant alkaloids, and toxins; and synthetic derivatives thereof. Exemplary compounds include, but are not limited to, alkylating agents: cisplatin, treosulfan, and trofosfamide; plant alkaloids: vinblastine, paclitaxel, docetaxol; DNA topoisomerase inhibitors: teniposide, crisnatol, and mitomycin; anti-folates: methotrexate, and mycophenolic acid; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents are also considered. For example, CHOP comprises cyclophosphamide, vincristine, doxorubicin, and prednisone. Additional examples of chemotherapeutic agents include, but are not limited to, temozolomide (TMZ), bendamustine, irinotecan, capecitabine, topotecan, camptothecin, cytarabine, fluorouracil, cyclophosphamide, etoposide phosphate, teniposide, doxorubicin, daunoaibicin, pemetrexed, mitomycin C, chlorambucil, and melphalan. The foregoing examples of chemotherapeutic agents are illustrative, and are not intended to be limiting.

In one embodiment, the chemotherapeutic agents are platinum compounds, such as cisplatin, carboplatin, oxaliplatin, nedaplatin, satraplatin, triplatin tetranitrate, and iproplatin. Other antineoplastic platinum coordination compounds are well known in the art, and can be modified according to well-known methods in the art, and include the compounds disclosed in U.S. Pat. Nos. 4,996,337, 4,946,954, 5,091,521, 5,434,256, 5,527,905, and 5,633,243, all of which are incorporated herein by reference.

In some embodiments, the chemotherapeutic agent is an alkylating agent. Alkylating agents can be classified into three groups: classical alkylating agents, alkylating-like agents, and non-classical alkylating agents. In contrast to the alkylating-like and non-classical agents, the classical alkylating agents include true alkyl groups, and work by impairing cell function by forming covalent bonds with the amino, carboxyl, sulfhydryl, and phosphate groups in biologically important molecules. Examples of classical alkylating agents include the nitrogen mustards, the nitrosoureas and the alkyl sulfonates. The ethylene imines (aziridines) and methyl melamines are also generally considered as classical, but can be considered non-classical as well. Specific examples of classical alkylating agents include: nitrogen mustards such as chlorambucil, chlornaphazine, cyclophosphamide (e.g., CYTOXAN®), estramustine, ifosfamide, mechlorethamine (mustine HN2), mechlorethamine oxide hydrochloride, melphalan, novembichin, phenesterine, prednimustine, trofosfamide, uracil mustard (uramustine) and mannomustards; nitrosoureas such as carmustine, chlorozotocin, fotemustine, lomustine and nimustine; alkyl sulfonates such as busulfan, improsulfan and piposulfan; ethylene imines (aziridines) and methyl melamines such as altretamine, triethylenemelamine, triethylenephosphoramide (TEPA), triethylenethiophosphoramide (e.g. ThioTEPA) and trimethylol melamine. Alkylating-like agents include platinum-based chemotherapeutic drugs (often referred to as platinum analogues). Although these agents do not have an alkyl group, they nevertheless damage DNA by permanently coordinating to DNA to interfere with DNA repair. Examples of platinum analogs include cisplatin (e.g. Carboquone), carboplatin, nedaplatin, oxiloplatin, oxaliplatin (Eloxatin™) and satraplatin. Some of the agents variously included in the non-classical alkylating category include procarbazine, triethylenethiophosphoramide (e.g., ThioTEPA) and its analogues such as altretamine (which are considered classical alkylating agents by some) and certain tetrazines such as dicarbazine and temozolomide.

In some embodiments, the alkylating agent is temozolomide (TMZ). The IUPAC name for TMZ is 4-methyl-5-oxo-2,3,4,6,8-pentazabicyclo[4.3.0]nona-2,7,9-triene-9-carboxamide. TMZ induces methylation of guanine at 06 position, a change that causes a futile cycle of attempted DNA repair, and results in cell apoptosis. In the United States, TMZ is available in 5 mg, 20 mg, 100 mg, 140 mg, 180 mg & 250 mg capsules or IV forms. TMZ is sold with trade names such as Temodar (manufactured by Merck), Temodal, Temcad. Without limitation, TMZ can be administered orally or intravenously.

In some embodiments, the DNA damaging agent is a PARP (e.g., PARP-1 and/or PARP-2) inhibitor and such inhibitors are well known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1,8-naphthalimide; (Trevigen); 6(5H)-phenanthridinone (Trevigen); benzamide (U.S. Pat. No. Re. 36,397); and NU1025 (Bowman et al.).

Ribonucleotide reductase inhibitors have been used as anti-cancer drugs to interfere with the growth of tumor cells by blocking the formation of deoxyribonucleotides. Deoxyribonucleotides are used in the synthesis of DNA. As used herein, the term “ribonucleotide reductase inhibitor” should be understood to encompass any agent capable of inhibiting (to any extent, i.e. qualitatively or quantitatively) the ribonucleotide reductase enzyme catalyzing the formation of deoxyribonucleotides from ribonucleotides.

Examples of ribonucleotide reductase inhibitors include, but are not limited to, fludarabine, cladribine, gemcitabine, tezacitabine, triapine, motexafm gadolinium, hydroxyurea, gallium maltolate, gallium nitrate, or any combination thereof. The ribonucleotide reductase inhibitor can include also any metabolites and prodrugs thereof. The ribonucleotide reductase inhibitor can also be a viral vector that inhibits ribonucleotide reductase. Examples of ribonucleotide reductase inhibitors are also disclosed in patents or applications such as U.S. Pat. No. 5,071,835, U.S. Pat. No. 5,672,586, US20090047250, WO2013116765, U.S. Pat. No. 7,427,605, and U.S. Pat. No. 4,814,432.

In some embodiments, the ribonucleotide reductase inhibitor is hydroxyurea. Also called hydroxycarbamide, hydroxyurea is sold with brand names such as Apo-Hydroxyurea, Droxia, and Hydrea.

In some embodiments, the ribonucleotide reductase inhibitor is fludarabine. The IUPAC name for fludarabine is [(2R,3R,4S,5R)-5-(6-amino-2-fluoro-purin-9-yl)- 3,4-dihydroxy-oxolan-2-yl]methoxyphosphonic acid. Fludarabine is sold with brand names such as Fludara and Oforta.

In some embodiments, the ribonucleotide reductase inhibitor is gemcitabine. The IUPAC name for gemcitabine is 4-amino-1-(2-deoxy-2,2-difluoro-β-D-erythro-pentofuranosyl)pyrimidin-2(1H)-on. Gemcitabine is sold as Gemzar by Eli Lilly and Company.

Methods for testing inhibition of ribonucleotide reductase activity are known to one skilled in the art and include, but are not limited to, in vitro or in vivo assays. For example, an in vitro assay is commercially available by NovoCIB. Exemplary methods are also disclosed in U.S. Pat. No. 5,834,279 and US20050255509.

In some embodiments, the DNA damaging agent is an alkylating agent and the ribonucleotide reductase inhibitor is hydroxyurea, fludarabine, gemcitabine, or a combination thereof.

In some embodiments, the DNA damaging agent is TMZ and the ribonucleotide reductase inhibitor is hydroxyurea, fludarabine, gemcitabine, or a combination thereof

In some embodiments, the DNA damaging agent is TMZ and the ribonucleotide reductase inhibitor is hydroxyurea.

In some embodiments, the DNA damaging agent is TMZ and the ribonucleotide reductase inhibitor is fludarabine.

In some embodiments, the DNA damaging agent is TMZ and the ribonucleotide reductase inhibitor is gemcitabine.

In some embodiments, the tumor is any tumor treatable by a DNA damaging agent.

In some embodiments, the tumor is selected from a group consisting of central nervous system (CNS) neoplasm, melanoma, recurrent adult acute lymphoblastic leukemia, recurrent childhood acute lymphoblastic leukemia, Ewing sarcoma, unspecified adult solid tumor, unspecified childhood solid tumor, hepatocellular carcinoma, pancreatic neuroendocrine tumor (e.g., gastrinoma, glucagonoma, insulinoma, islet cell carcinoma, pancreatic polypeptide tumor, recurrent islet cell carcinoma, or somatostatinoma), lung cancer, colorectal cancer, rectal cancer, breast cancer, ovarian cancer, rhabdomyosarcoma, acute myelogenous leukemia, and myelodysplastic syndrome.

The CNS neoplasm can be brain tumor, glioma, glioblastoma, oligodendroglioma, astrocytoma, medulloblastoma, oligoastrocytoma, gliosarcoma, recurrent adult brain tumor, B-cell lymphoma originating in the CNS, childhood high-grade cerebellar astrocytoma, childhood high-grade cerebral astrocytoma, childhood spinal cord neoplasm, childhood brain stem glioma, childhood cerebral astrocytoma, peripheral primitive neuroectodermal tumor, recurrent childhood medulloblastoma, recurrent childhood supratentorial primitive neuroectodermal tumor, or recurrent childhood pineoblastoma.

In some embodiments, the tumor is glioblastoma (also known as Grade IV astrocytoma), melanoma, relapsed Grade III anaplastic astrocytoma, oligodendroglioma, prolactinoma, or relapsed primary CNS lymphoma.

In some embodiments, the subject to be treated has been diagnosed as having a tumor known to develop resistance to the DNA damaging agent.

In some embodiments, the subject to be treated is diagnosed as having a recurrent tumor such as recurrent glioblastoma.

In some embodiments, the tumor has developed resistance to the DNA damaging agent. For example, the tumor is TMZ-resistant glioblastoma. In these embodiments, the ribonucleotide reductase inhibitor being administered can sensitize the tumor cells to the DNA damaging agent. It should be noted, however, that the DNA damaging agent being administered need not be a DNA damaging agent to which the tumor has developed resistance.

In some embodiments, the tumor has a propensity to develop resistance to a particular DNA damaging agent.

Methods to determine whether a tumor is resistant to the DNA damaging agent are well known in the art. These methods include, but are not limited to, fresh tumor cell culture tests, cancer biomarker tests, and positron emission tomography (PET) tests. See, for example, Lippert et al., Int. J. Med. Sci. 2011, 8, 245-253.

In some embodiments, the method further comprises providing the subject at least one (e.g., 1, 2, 3, 4, 5, or more) other anti-cancer treatment. In some embodiments, the one other anti-cancer treatment is radiation therapy. The dose of radiation varies depending on the type and stage of tumor being treated, and whether the patient is receiving chemotherapy. The dose of radiation can be 5-100 Gy, 10 to 80 Gy, 20 to 80 Gy, 20 to 60 Gy, or 10 to 60 Gy. In some embodiment, the one other anti-cancer treatment comprises other chemotherapeutic agent or PARD inhibitor.

In some embodiments, the subject is a mammal.

In some embodiments, the subject is a human.

The DNA damaging agent and the ribonucleotide reductase inhibitor may be administered in any dose or dosing regimen. Specific dosage and treatment regimens for any particular subject will depend upon a variety of factors, including, but not limited to, age, body weight, general health status, gender, diet, time of administration, rate of excretion, drug combination, the severity and course of the tumor, the type of the tumor, and the judgment of the physician.

With respect to the therapeutic methods of the invention, it is not intended that the administration be limited to a particular mode of administration, dosage, or frequency of dosing. An effective amount, e.g., a therapeutically effective dose of the agent disclosed herein may be administered to the patient in a single dose or in multiple doses. When multiple doses are administered, the doses may be separated from one another by, for example, one hour, three hours, six hours, eight hours, one day, two days, one week, two weeks, or one month. For example, the DNA damaging agent and the ribonucleotide reductase inhibitor can each be administered for, e.g., 2, 3, 4, 5, 6, 7, 8, 10, 15, 20, or more weeks. It is to be understood that, for any particular subject, specific dosage regimes should be adjusted over time according to the individual need and the professional judgment of the person administering or supervising the administration of the compositions. For example, the dosage of the therapeutic can be increased if the lower dose does not provide sufficient therapeutic activity.

The dosage can be determined by one of skill in the art and can also be adjusted by the individual physician in the event of any complication. Typically, the dosage of the DNA damaging agent and the ribonucleotide reductase inhibitor can each range from 0.001 mg/kg body weight to 5 g/kg body weight. In some embodiments, the dosage range is from 0.001 mg/kg body weight to lg/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, or from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, or from 4.5 g/kg body weight to 5 g/kg body weight. Effective doses may be extrapolated from dose-response curves derived from in vitro or animal model test bioassays or systems. The dosage should not be so large as to cause unacceptable adverse side effects.

In some embodiments, the DNA damaging agent and the ribonucleotide reductase inhibitor can each be administered in a dose of from about 20 mg/m² to about 5,000 mg/m² body surface area. For example, the dose can be from about 20 mg/m² to about 200 mg/m² body surface area; the dose can be from about 150 mg/m² to about 500 mg/m² body surface area; the dose can be from about 400 mg/m² to about 1000 mg/m² body surface area; the dose can be from about 900 mg/m² to about 5,000 mg/m² body surface area; the dose can be from about 200 mg/m² to about 1,000 mg/m² body surface area; or the dose can be from about 500 mg/m² to about 600 mg/m² body surface area.

The current standard dosage of the DNA damaging agent can also serve as a guideline for the dosage used in the method described herein. Current standard dosages for a variety of DNA damaging agents are readily available information. For example, the standard dosage of cisplatin is 50 mg/m² IV or more every 3 week; or 20 mg/m² IV daily for 4-5 days every 3-4 week. In another example, the standard initial dosage of TMZ for treating glioblastoma multiforme in an adult is 75 mg/m² daily either orally or by intravenous infusion over 90 minutes for 42 days concomitant with focal radiotherapy. It should be noted that, in the methods described herein, because the ribonucleotide reductase inhibitor can sensitize tumor cells to the DNA damaging agent, it may be possible to use a lower dosage of the DNA damaging agent than the present standard dosage, while achieving the same or better efficacy.

The physician can determine the ratio of the DNA damaging agent to the ribonucleotide reductase inhibitor so that it is sufficient for the ribonucleotide reductase inhibitor to sensitize tumor cells to the DNA damaging agent. The ratio of the DNA damaging agent to the ribonucleotide reductase inhibitor can be determined by, for example, in vitro studies using tumor cell lines (e.g., tumor cells resistant to the DNA damaging agent), or in vivo studies using animal models such as mice. By varying the ratio and monitoring the resultant therapeutic effects, a skilled artisan can readily determine the optimal ratio without undue experimentation.

The molar ratio of the DNA damaging agent over the ribonucleotide reductase inhibitor can be from 0.001:1 to 1000:1, from 0.001:1 to 500:1, from 0.001:1 to 250:1, from 0.001:1 to 100:1, from 0.001:1 to 50:1, from 0.001:1 to 10:1, from 0.01:1 to 1000:1, from 0.01:1 to 500:1, from 0.01:1 to 250:1, from 0.01:1 to 100:1, from 0.01:1 to 50:1, from 0.01:1 to 10:1, from 0.1:1 to 1000:1, from 0.1:1 to 500:1, from 0.1:1 to 250:1, from 0.1:1 to 100:1, from 0.1:1 to 50:1, from 0.1:1 to 10:1, from 1:1 to 1000:1, from 1:1 to 500:1, from 1:1 to 250:1, from 1:1 to 100:1, from 1:1 to 50:1, or from 1:1 to 10:1. It should be noted that the molar ratio can depend on factors such as the type of tumor being treated, the severity of the tumor, the subject being treated (e.g., gender, race, or age), the particular type of the DNA damaging agent, and the particular type of the ribonucleotide reductase inhibitor.

A physician may, for example, prescribe a relatively low dose at first, subsequently increasing the dose until an appropriate response is obtained. The dose administered to a patient is sufficient to effect a beneficial therapeutic response in the patient over time, or, e.g., to reduce symptoms, or other appropriate activity, depending on the application. The dose is determined by the efficacy of the particular formulation, and the activity, stability or serum half-life of the composition being administered, and the condition of the patient, the particular tumor to be treated, as well as the body weight or body surface area. The size of the dose is also determined by the existence, nature, and extent of any adverse side- effects that accompany the administration of a particular formulation, or the like in a particular subject. Therapeutic compositions are optionally tested in one or more appropriate in vitro and/or in vivo animal models of disease, and known to persons of ordinary skill in the art, to confirm efficacy, tissue metabolism, and to estimate dosages, according to methods well known in the art. In particular, dosages can be initially determined by activity, stability or other suitable measures of treatment vs. non-treatment (e.g., comparison of treated vs. untreated cells or animal models), in a relevant assay. Formulations are administered at a rate determined by the LD50 of the relevant formulation, and/or observation of any side-effects of the pharmaceutical composition at various concentrations, e.g., as applied to the mass and overall health of the patient. Administration can be accomplished via single or divided doses.

For example, a therapeutically effective amount can be estimated initially either in cell culture assays or in animal models, usually mice, rabbits, dogs, or pigs. The animal model is also used to achieve a desirable concentration range and route of administration. Such information can then be used to determine useful doses and routes for administration in other subjects. Generally, the therapeutically effective amount is dependent on the desired therapeutic effect. In certain embodiments, the therapeutic effect is reduction in tumor size by a statistically significant amount. In some embodiments, tumor size is reduced by at least 10%, at least 20%, at least 30%, at least 40%, at least 50%, at least 60%, at least 70%, at least 80%, or more.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art; e.g., in Remington's Pharmaceutical Sciences (Mack Pub. Co. 17^{th} ed.), above.

It should also be noted that the DNA damaging agent and the ribonucleotide reductase inhibitor can be administered in the same route or in different routes. For example, the DNA damaging agent is administered orally or intravenously, while and the ribonucleotide reductase inhibitor is administered orally or intravenously.

Another aspect of the invention relates to a pharmaceutical composition comprising a DNA damaging agent and a ribonucleotide reductase inhibitor, in which the DNA damaging agent is different from the ribonucleotide reductase inhibitor. While the DNA damaging agent and the ribonucleotide reductase inhibitor can be administered in two different compositions, a composition comprising them together can yield advantages such as convenience and better patient compliance. A composition comprising both the DNA damaging agent and the ribonucleotide reductase inhibitor is particularly useful for treatment methods that require the simultaneous administration of these two compounds.

In some embodiments, the DNA damaging agent and the ribonucleotide reductase inhibitor is in a ratio sufficient for the ribonucleotide reductase inhibitor to sensitize tumor cells to the DNA damaging agent. The ratio of the DNA damaging agent to the ribonucleotide reductase inhibitor can be determined by, for example, in vitro studies using tumor cell lines (e.g., tumor cells resistant to the DNA damaging agent), or in vivo studies using animal models such as mice. By varying the ratio and monitoring the resultant therapeutic effects, a skilled artisan can readily determine the optimal ratio without undue experimentation.

The molar ratio of the DNA damaging agent over the ribonucleotide reductase inhibitor can be from 0.001:1 to 1000:1, from 0.001:1 to 500:1, from 0.001:1 to 250:1, from 0.001:1 to 100:1, from 0.001:1 to 50:1, from 0.001:1 to 10:1, from 0.01:1 to 1000:1, from 0.01:1 to 500:1, from 0.01:1 to 250:1, from 0.01:1 to 100:1, from 0.01:1 to 50:1, from 0.01:1 to 10:1, from 0.1:1 to 1000:1, from 0.1:1 to 500:1, from 0.1:1 to 250:1, from 0.1:1 to 100:1, from 0.1:1 to 50:1, from 0.1:1 to 10:1, from 1:1 to 1000:1, from 1:1 to 500:1, from 1:1 to 250:1, from 1:1 to 100:1, from 1:1 to 50:1, or from 1:1 to 10:1. It should be noted that the molar ratio can depend on factors such as the type of tumor being treated, the severity of the tumor, the subject being treated (e.g., gender, race, or age), the particular type of the DNA damaging agent, and the particular type of the ribonucleotide reductase inhibitor. In some embodiments, the molar ratio is sufficient to ameliorate one or more symptoms in the subject caused by the tumor, e.g., headaches, nausea, and seizures in the case of glioma. In certain embodiments, the molar ratio is sufficient to reduce the tumor size, e.g., as measured by estimate tumor volume.

In some embodiments, the composition described herein further comprises a pharmaceutically-acceptable carrier.

In some embodiments, the composition described herein can be formulated in a unit dosage injectable form (e.g., solution, suspension, emulsion). The pharmaceutical formulations suitable for injection include sterile aqueous solutions or dispersions and sterile powders for reconstitution into sterile injectable solutions or dispersions. The term “dosage unit” form as used herein refers to physically discrete units suited as unitary dosages for the subjects to be treated; each unit containing a predetermined quantity of active compound calculated to produce the desired therapeutic effect in association with the required pharmaceutical carrier.

In some embodiments, the composition described herein can be used for the preparation of a medicament for the treatment of tumor.

It should be understood that this invention is not limited to the particular methodology, protocols, and reagents, etc., disclosed herein and as such may vary. The terminology used herein is for the purpose of describing particular embodiments only, and is not intended to limit the scope of the present invention, which is defined solely by the claims.

As used herein and in the claims, the singular forms include the plural reference and vice versa unless the context clearly indicates otherwise. Other than in the operating examples, or where otherwise indicated, all numbers expressing quantities of ingredients or reaction conditions used herein should be understood as modified in all instances by the term “about.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Although any known methods, devices, and materials may be used in the practice or testing of the invention, the methods, devices, and materials in this regard are disclosed herein.

Some embodiments of the invention are listed in the following paragraphs:

Paragraph 1. A method of treating a tumor in a subject, the method comprising administering to the subject a therapeutically effective amount of a DNA damaging agent and a ribonucleotide reductase inhibitor.
Paragraph 2. The method of paragraph 1, wherein the ribonucleotide reductase inhibitor is selected from a group consisting of hydroxyurea, motexafin gadolinium, fludarabine, cladribine, gemcitabine, tezacitabine, triapine, gallium maltolate, gallium nitrate, and a combination thereof.
Paragraph 3. The method of paragraph 1, wherein the ribonucleotide reductase inhibitor is hydroxyurea, fludarabine, gemcitabine, or a combination thereof.
Paragraph 4. The method of paragraph 1, wherein the DNA damaging agent is not a ribonucleotide reductase inhibitor.
Paragraph 5. The method of paragraph 1, wherein the DNA damaging agent is not radiation.
Paragraph 6. The method of any of paragraphs 1-5, wherein the DNA damaging agent is a chemotherapeutic agent or a PARP inhibitor.
Paragraph 7. The method of paragraph 6, wherein the chemotherapeutic agent is selected from a group consisting of temozolomide (TMZ), bendamustine, irinotecan, capecitabine, topotecan, cisplatin, oxaliplatin, carboplatin, nedaplatin, satraplatin, triplatin tetranitrate, camptothecin, cytarabine, fluorouracil, cyclophosphamide, etoposide phosphate, teniposide, doxorubicin, daunoaibicin, pemetrexed, mitomycin C, chlorambucil, and melphalan.
Paragraph 8. The method of paragraph 7, wherein the chemotherapeutic agent is temozolomide (TMZ).
Paragraph 9. The method of any of paragraphs 1-8, wherein the ratio of the DNA damaging agent to the ribonucleotide reductase inhibitor is sufficient to sensitize tumor cells to the DNA damaging agent.
Paragraph 10. The method of any of paragraphs 1-9, wherein the tumor is selected from a group consisting of central nervous system (CNS) neoplasm, melanoma, recurrent adult acute lymphoblastic leukemia, recurrent childhood acute lymphoblastic leukemia, Ewing sarcoma, unspecified adult solid tumor, unspecified childhood solid tumor, hepatocellular carcinoma, pancreatic neuroendocrine tumor (e.g., gastrinoma, glucagonoma, insulinoma, islet cell carcinoma, pancreatic polypeptide tumor, recurrent islet cell carcinoma, or somatostatinoma), lung cancer, colorectal cancer, rectal cancer, breast cancer, ovarian cancer, rhabdomyosarcoma, acute myelogenous leukemia, and myelodysplastic syndrome.
Paragraph 11. The method of paragraph 10, wherein the CNS neoplasm is selected from a group consisting of glioma, glioblastoma, oligodendroglioma, astrocytoma, medulloblastoma, oligoastrocytoma, gliosarcoma, recurrent adult brain tumor, B-cell lymphoma originating in the CNS, childhood high-grade cerebellar astrocytoma, childhood high-grade cerebral astrocytoma, childhood spinal cord neoplasm, childhood brain stem glioma, childhood cerebral astrocytoma, peripheral primitive neuroectodermal tumor, recurrent childhood medulloblastoma, recurrent childhood supratentorial primitive neuroectodermal tumor, and recurrent childhood pineoblastoma.
Paragraph 12. The method of paragraph 1, wherein the tumor is glioblastoma or melanoma.
Paragraph 13. The method of paragraph 12, wherein the glioblastoma is recurrent glioblastoma.
Paragraph 14. The method of any of paragraphs 1-13, wherein the tumor is resistant to the DNA damaging agent.
Paragraph 15. The method of any of paragraphs 1-14, wherein the ribonucleotide reductase inhibitor is administered before the administration of the DNA damaging agent.
Paragraph 16. The method of any of paragraphs 1-14, wherein the ribonucleotide reductase inhibitor is administered simultaneously with the administration of the DNA damaging agent.
Paragraph 17. The method of any of paragraphs 1-14, wherein the ribonucleotide reductase inhibitor is administered after the administration of the DNA damaging agent.
Paragraph 18. The method of any of paragraphs 1-17, further comprising providing the subject at least one other anti-cancer treatment.
Paragraph 19. The method of paragraph 18, wherein the one other anti-cancer treatment is radiation therapy.
Paragraph 20. The method of any of paragraphs 1-19, wherein the subject is a mammal.
Paragraph 21. The method of paragraph 20, wherein the subject is a human.
Paragraph 22. Use of a ribonucleotide reductase inhibitor in combination with a DNA damaging agent for the preparation of a medicament for the treatment of tumor, wherein the DNA damaging agent is not a ribonucleotide reductase inhibitor.
Paragraph 23. The use of paragraph 22, wherein the ribonucleotide reductase inhibitor is selected from a group consisting of hydroxyurea, motexafin gadolinium, fludarabine, cladribine, gemcitabine, tezacitabine, triapine, gallium maltolate, and gallium nitrate, and a combination thereof.
Paragraph 24. The use of paragraph 22 or 23, wherein the DNA damaging agent is a chemotherapeutic agent or a PARP inhibitor.
Paragraph 25. The use of paragraph 24, wherein the chemotherapeutic agent is selected from a group consisting of temozolomide (TMZ), bendamustine, irinotecan, capecitabine, topotecan, cisplatin, oxaliplatin, carboplatin, nedaplatin, satraplatin, triplatin tetranitrate, camptothecin, cytarabine, fluorouracil, cyclophosphamide, etoposide phosphate, teniposide, doxorubicin, daunoaibicin, pemetrexed, mitomycin C, chlorambucil, and melphalan.
Paragraph 26. The use of paragraph 25, wherein the chemotherapeutic agent is temozolomide (TMZ).
Paragraph 27. A pharmaceutical composition comprising an effective amount of a DNA damaging agent and a ribonucleotide reductase inhibitor.
Paragraph 28. The pharmaceutical composition of paragraph 27, wherein the DNA damaging agent is not a ribonucleotide reductase inhibitor.
Paragraph 29. The pharmaceutical composition of paragraph 27 or 28, wherein the DNA damaging agent and the ribonucleotide reductase inhibitor is in a ratio sufficient for the ribonucleotide reductase inhibitor to sensitize tumor cells to the DNA damaging agent.
Paragraph 30. The pharmaceutical composition of any of paragraphs 27-29, wherein the ribonucleotide reductase inhibitor is selected from a group consisting of hydroxyurea, motexafin gadolinium, fludarabine, cladribine, gemcitabine, tezacitabine, triapine, gallium maltolate, gallium nitrate, and a combination thereof.
Paragraph 31. The pharmaceutical composition of paragraph 30, wherein the ribonucleotide reductase inhibitor is hydroxyurea, fludarabine, gemcitabine, or a combination thereof.
Paragraph 32. The pharmaceutical composition of any of paragraphs 27-31, wherein the DNA damaging agent is a chemotherapeutic agent or a PARP inhibitor.
Paragraph 33. The pharmaceutical composition of paragraph 32, wherein the chemotherapeutic agent is selected from a group consisting of temozolomide (TMZ), bendamustine, irinotecan, capecitabine, topotecan, cisplatin, oxaliplatin, carboplatin, nedaplatin, satraplatin, triplatin tetranitrate, camptothecin, cytarabine, fluorouracil, cyclophosphamide, etoposide phosphate, teniposide, doxorubicin, daunoaibicin. pemetrexed, mitomycin C, chlorambucil, and melphalan.
Paragraph 34. The pharmaceutical composition of paragraph 33, wherein the chemotherapeutic agent is temozolomide (TMZ).

### Definitions

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

As used herein the term “comprising” or “comprises” is used in reference to compositions, methods, and respective component(s) thereof, that are useful to an embodiment, yet open to the inclusion of unspecified elements, whether useful or not.

As used herein the term “consisting essentially of” refers to those elements required for a given embodiment. The term permits the presence of elements that do not materially affect the basic and novel or functional characteristic(s) of that embodiment of the invention.

As used herein, the term “cancer” in a subject refers to the presence of cells possessing characteristics typical of cancer-causing cells, such as uncontrolled proliferation, immortality, metastatic potential, rapid growth and proliferation rate, loss of contact inhibition and certain characteristic morphological features. Often, cancer cells will be in the form of a tumor, but such cells may exist alone within a subject, or may be a non-tumorigenic cancer cell, such as a leukemia cell. Examples of cancer include but are not limited to breast cancer, melanoma, adrenal gland cancer, biliary tract cancer, bladder cancer, brain or central nervous system cancer, bronchus cancer, blastoma, carcinoma, a chondrosarcoma, cancer of the oral cavity or pharynx, cervical cancer, colon cancer, colorectal cancer, esophageal cancer, gastrointestinal cancer, glioblastoma, hepatic carcinoma, hepatoma, kidney cancer, LAM, leukemia, liver cancer, lung cancer, lymphoma, non-small cell lung cancer, osteosarcoma, ovarian cancer, pancreas cancer, peripheral nervous system cancer, prostate cancer, sarcoma, salivary gland cancer, small bowel or appendix cancer, small-cell lung cancer, squamous cell cancer, stomach cancer, testis cancer, thyroid cancer, TSC, urinary bladder cancer, uterine or endometrial cancer, and vulval cancer.

As used herein, the term “tumor” means a mass of transformed cells that are characterized by neoplastic uncontrolled cell multiplication and at least in part, by containing angiogenic vasculature. The abnormal neoplastic cell growth is rapid and continues even after the stimuli that initiated the new growth has ceased. The term “tumor” is used broadly to include the tumor parenchymal cells as well as the supporting stroma, including the angiogenic blood vessels that infiltrate the tumor parenchymal cell mass. Although a tumor generally is a malignant tumor, i.e., a cancer having the ability to metastasize (i.e., a metastatic tumor), a tumor also can be nonmalignant (i.e., non-metastatic tumor). Tumors are hallmarks of cancer, a neoplastic disease the natural course of which is fatal. Cancer cells exhibit the properties of invasion and metastasis and are highly anaplastic.

As used herein, the term “sensitize” or the phrase “increase sensitivity or responsiveness to” a DNA damaging agent means to alter cancer cells or tumor cells in a way that allows for more effective treatment (e.g., killing or preventing the growth of cancerous cells, tumor size reduction, and/or prolonged survival of the subject) of the associated neoplastic disease with the DNA damaging agent. For example, providing a cell or subject with an effective amount of an agent that inhibits ribonucleotide reductase sensitizes a tumor cell, previously resistant, to treatment with a DNA damaging agent.

As used herein, the terms “resistant” or “resistance” to a therapeutic agent such as a DNA damaging agent mean that the therapeutic agent fails to achieve the intended therapeutic effects (e.g., killing or preventing the growth of cancerous cells) in the subject being treated, or that the tumor is no longer responsive to the therapeutic agent. Resistance to the therapeutic agent can be intrinsic or acquired.

As used herein, the term “responsive” means a desired reaction of a cell, organism or subject to treatment with a therapeutic agent such as a DNA damaging agent.

As used herein, the term “recurrent” when used with a tumor, refers to a tumor, for example, glioma, that has come back after treatment, usually after a period of time during which the tumor could not be detected. The tumor may come back to the same place or to another place in the body of a subject.

As used herein, the term “agent” refers to any kind of compound, molecule or ion, or any combination thereof. An agent can be an organic molecule, an inorganic molecule, a biological molecule or analog thereof

As used herein, the term “inhibitor” refers to an agent that can decrease the function or activity of a biological molecule. For example, a ribonucleotide reductase inhibitor can decrease the function or activity of ribonucleotide reductase by at least 5%, at least 10%, at least 20%, at least 30%, at least 40%, at least 50%, at least 60%, at least 70%, at least 80%, at least 90%, or at least 95%.

As used herein, the term “administer” refers to the placement of a composition into a subject by a method or route which results in at least partial localization of the composition at a desired site such that desired effect is produced. A compound or composition disclosed herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments, the compositions are administered by intravenous infusion or injection.

As used herein, the phrase “therapeutically-effective amount” or “effective amount” means that amount of a DNA damaging agent, a ribonucleotide reductase inhibitor, or a combination thereof, which is effective for producing some desired therapeutic effect in at least a sub-population of cells in a subject at a reasonable benefit/risk ratio applicable to any medical treatment. For example, an amount of a DNA damaging agent and a ribonucleotide reductase inhibitor administered to a subject that is sufficient to produce a statistically significant, measurable change in at least one symptom of a tumor (e.g., tumor size reduction).

As used herein, the terms “treat”, “treatment”, or “treating” refer to therapeutic treatment, wherein the objective is to slow down (lessen) an undesired physiological change or disorder, such as the progression of cancer. Beneficial or desired clinical results can include, but are not limited to, tumor size reduction, reduction of the metastatic potential of the tumor, alleviation of symptoms, diminishment of extent of tumor, stabilized (i.e., not worsening) state of tumor, delay or slowing of tumor progression, amelioration or palliation of tumor, and remission (whether partial or total), whether detectable or undetectable. Any particular treatment regimen can provide one or more such clinical results in one or more patients, and need not provide all such clinical results. “Treatment” can also mean prolonging survival as compared to expected survival if not receiving treatment.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “patient” and “subject” are used interchangeably herein. The terms, “patient” and “subject” are used interchangeably herein.

Preferably, the subject is a mammal. The mammal can be a human, non-human primate, mouse, rat, dog, cat, horse, or cow, but are not limited to these examples. Mammals other than humans can be advantageously used as subjects that represent animal models of tumors.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

As used herein, the term “statistically significant” refers to statistical significance and generally means a two standard deviation (2SD) below normal, or lower, than a reference value. The term refers to statistical evidence that there is a difference. It is defined as the probability of making a decision to reject the null hypothesis when the null hypothesis is actually true. The decision is often made using the p-value.

The singular terms “a,” “an,” and “the” include plural referents unless context clearly indicates otherwise. Similarly, the word “or” is intended to include “and” unless the context clearly indicates otherwise.

Other than in the operating examples, or where otherwise indicated, all numbers expressing quantities of ingredients or reaction conditions used herein should be understood as modified in all instances by the term “about.” The term “about” when used in connection with percentages may mean ±1% of the value being referred to. For example, about 100 means from 99 to 101.

Although methods and materials similar or equivalent to those disclosed herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology disclosed herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are disclosed herein for illustrative purposes, various equivalent modifications are possible within the scope of the disclosure, as those skilled in the relevant art will recognize For example, while method steps or functions are presented in a given order, alternative embodiments may perform functions in a different order, or functions may be performed substantially concurrently. The teachings of the disclosure provided herein can be applied to other procedures or methods as appropriate. The various embodiments disclosed herein can be combined to provide further embodiments. Aspects of the disclosure can be modified, if necessary, to employ the compositions, functions and concepts of the above references and application to provide yet further embodiments of the disclosure.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### EXAMPLES

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

The technology disclosed herein is further illustrated by the following examples which in no way should be construed as being further limiting.

### Example

### Hydroxyurea Sensitizes Glioblastoma to temozolomide

Hydroxyurea (HU), an FDA-approved drug, has been identified to sensitize TMZ-resistant GBM cells to temozolomide. HU was evaluated for the treatment of both newly diagnosed GBM as well as recurrent, TMZ-resistant, tumors. The inventors employed cells obtained from patient tumor tissues with different MGMT status and genetic modifications, which after intracranial injection infiltrate the brain of mice similar to GBM in patients. HU synergizes with TMZ, on both newly diagnosed and recurrent tumors, irrespective of the MGMT promoter methylation status. Although HU has been previously evaluated in malignant gliomas in combination with radiation or cytotoxic chemotherapy, and has shown limited efficacy, it was never evaluated in combination with TMZ. Here preclinical efficacy and safety of HU in combination with TMZ were demonstrated. HU sensitizes resistant glioma cells to TMZ in vitro

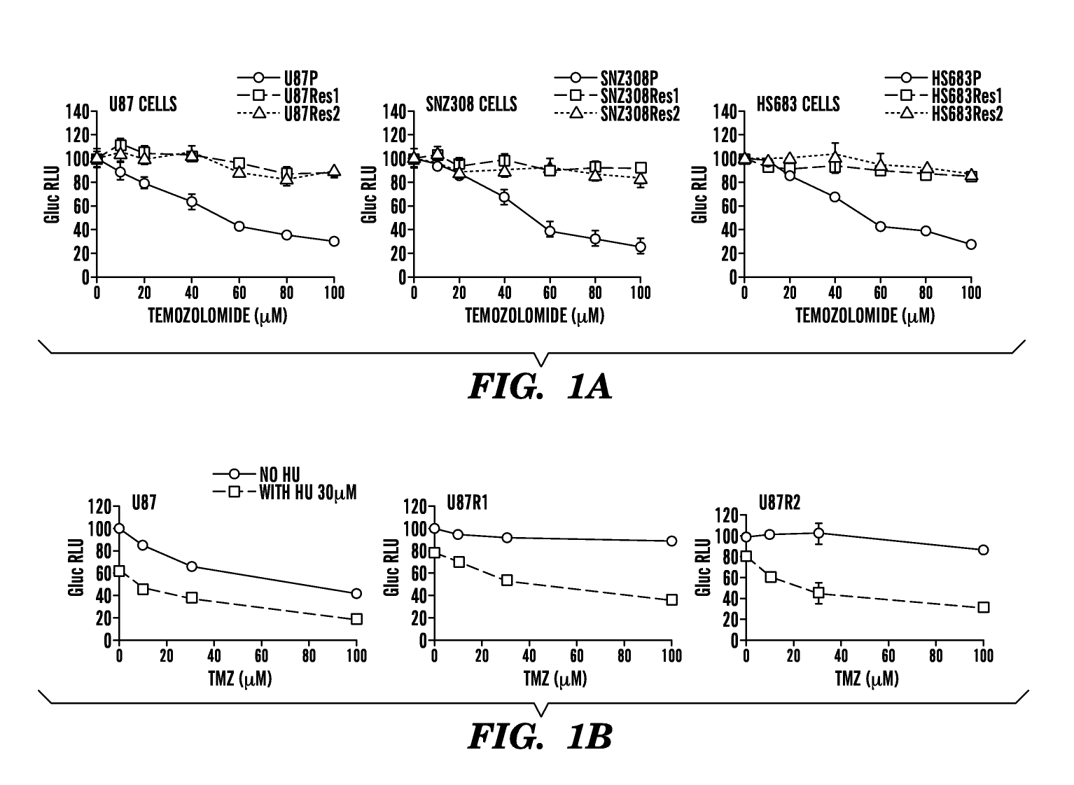

The inventors previously generated two independent TMZ-resistant subclones from three different glioma cell lines (U87, SNZ308 and HS683) by long-term exposure (two times per week over seven weeks) to 100 μM TMZ, generating U87R1, U87R2, SNZ308R1, SNZ308R2, HS683R1 and HS683R2. These cells together with their parental counterparts were first engineered by lentivirus vector transduction to stably express the naturally secreted Gaussia luciferase (Gluc) as a reporter for cell viability. It was previously shown that the level of Gluc secretion to the conditioned medium is linearly related with respect to cell number and proliferation (Tannous, B. A., Nat Protoc 2009, 4, 582-591; Tannous, B. A., et al., Mol Ther 2005, 11, 435-443; Wurdinger, T., et al., Nat Methods 2008, 5, 171-173). In this case, cell viability can be monitored over time by assaying aliquots of conditioned medium from these cells for Gluc activity. Exposure of these cells to TMZ revealed that the three parental glioma lines were sensitive to TMZ in a dose-dependent fashion, while no significant effect was observed on any of the resistant cell lines up to 100 μM (FIG. 1A). Using the U87R1 and U87R2 TMZ-resistant cells and the Gluc high-throughput screening assay (Badr, C. E., et al., Assay Drug Dev Technol, 2011 9, 281-289), a small library of 100 known anti-cancer agents was screened in the presence and absence of 100 μM TMZ. After screening and validation, the compound HU was found to sensitize both resistance cell lines to TMZ (FIG. 1B). The same results were obtained when HU was used in combination with TMZ on SNZ308 and HS683 resistant lines (data not shown).

### HU Sensitizes Resistant Glioma Cells to TMZ In Vivo

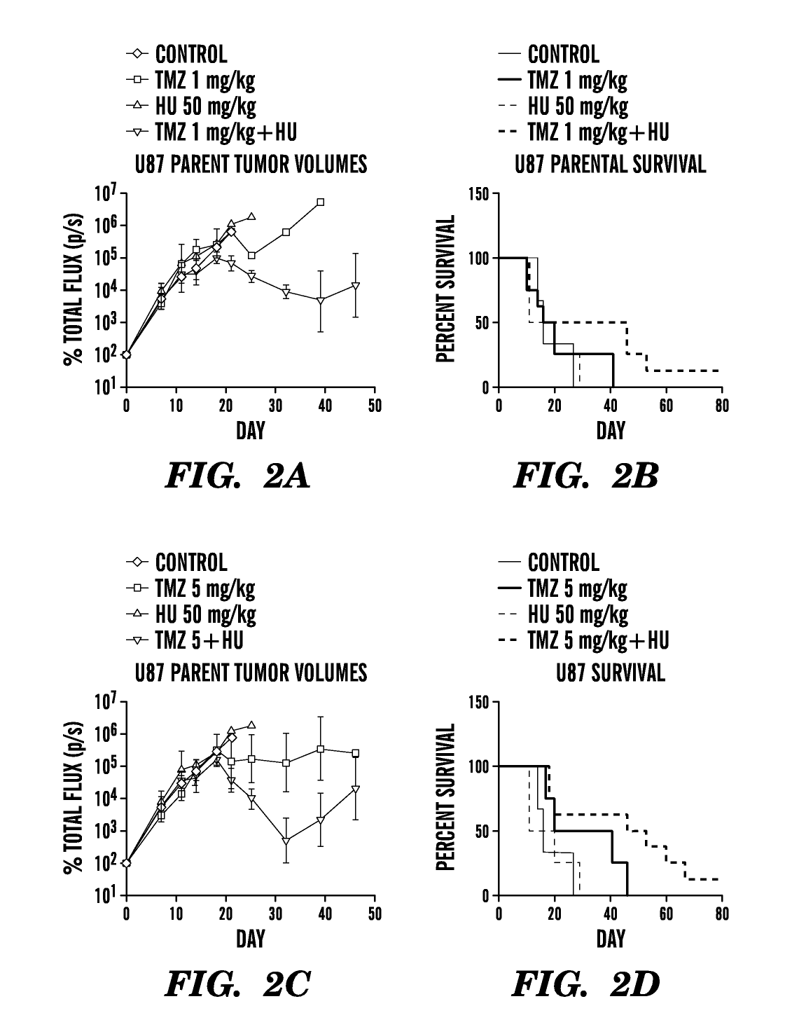

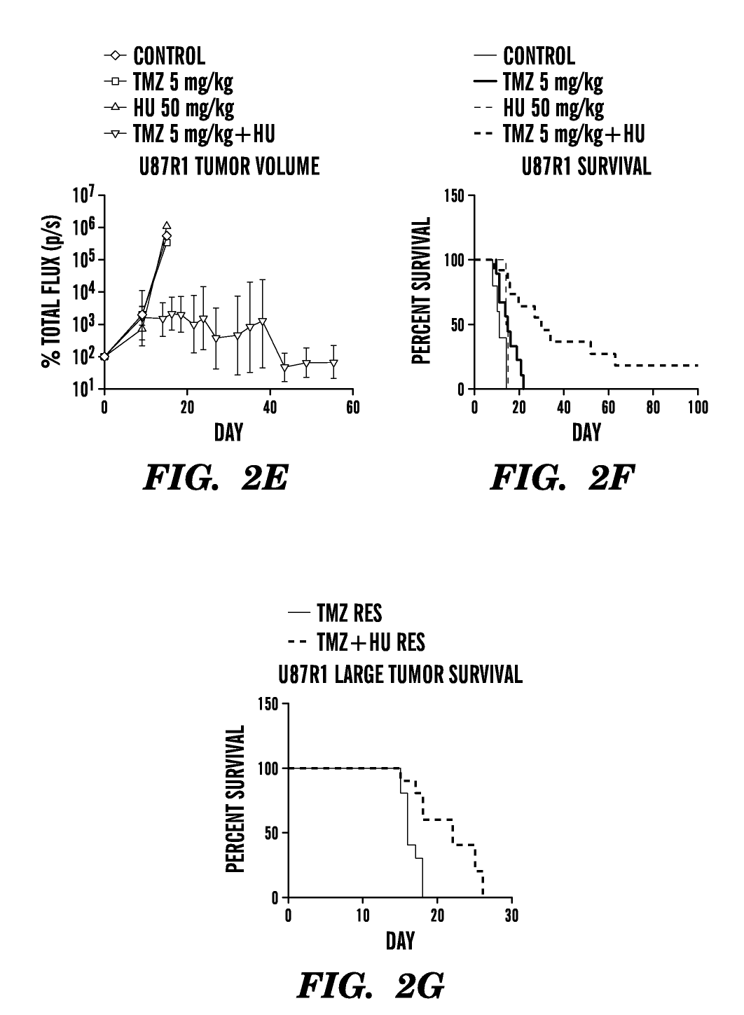

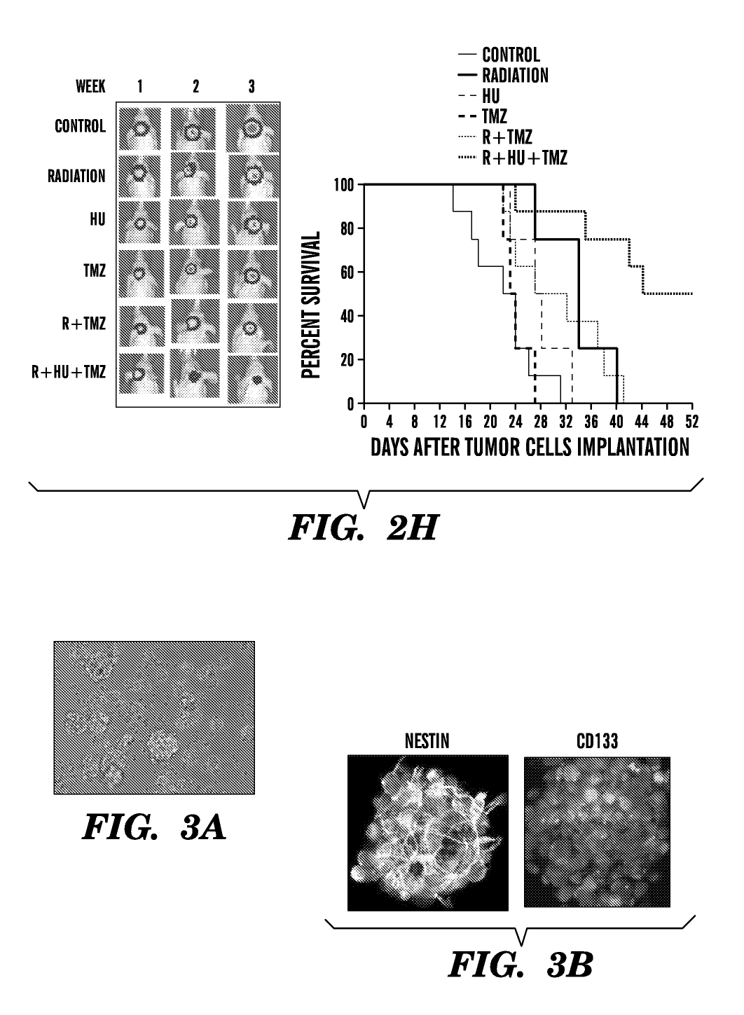

These data were then validated in an in vivo intracranial model. U87 or U87R1 cells were first transduced with a lentivirus vector to stably express firefly luciferase (Fluc), a reporter which can be used to track tumor growth in vivo (Wurdinger, T., et al., Nat Methods 2008, 5, 171-173). Fifty thousands of these cells were intracranially injected in the brain of nude mice as previously described. One week post-tumor implantation, mice were randomized into four different groups receiving: (1) DMSO vehicle control; (2) intraperitoneal (i.p) injection of 5 mg/kg TMZ; (3) 50 mg/kg HU i.p.; (4) combination of TMZ+HU. Tumor growth was monitored over time by in vivo Fluc bioluminescence imaging using a cooled CCD camera (Wurdinger, T., et al., Nat Methods 2008, 5, 171-173). HU alone had no significant effect on U87 or U87R1 tumor growth and mice survival. TMZ alone had a moderate effect on parental U87 tumors and survival rate but not on U87R1 tumors. On the other hand, combination of both HU+TMZ slowed down both U87 and U87R1 tumors leading to a significant increase in mice survival (FIGS. 2A-2F). In another experiment, this therapeutic strategy was challenged by implanting higher number of U87R1 cells (200,000 cells) and waiting two weeks before starting the combined therapy to allow the tumor to grow to a large size. Based on Fluc imaging, tumors had reached saturation signal and mice were expected to die within few days. As expected, all control mice died within 2-4 days while the HU/TMZ-treated mice survived for another 10 days showing the efficiency of this combined therapy (FIG. 2G). The effect of HU on U87R1 cells was then tested in combination with standard of care (radiation and TMZ) using the same intracranial model (50,000 U87R1 cells implanted and therapy initiated one week later). The triple therapy yielded an enhanced therapeutic effect on U87R1 tumors and increased survival rate with 50% of mice remained alive 52 days after implantation of the GBM cells (FIG. 2H).

### HU Sensitizes Primary GBM Cells to TMZ Irrespective of the MGMT Promoter Methylation Status

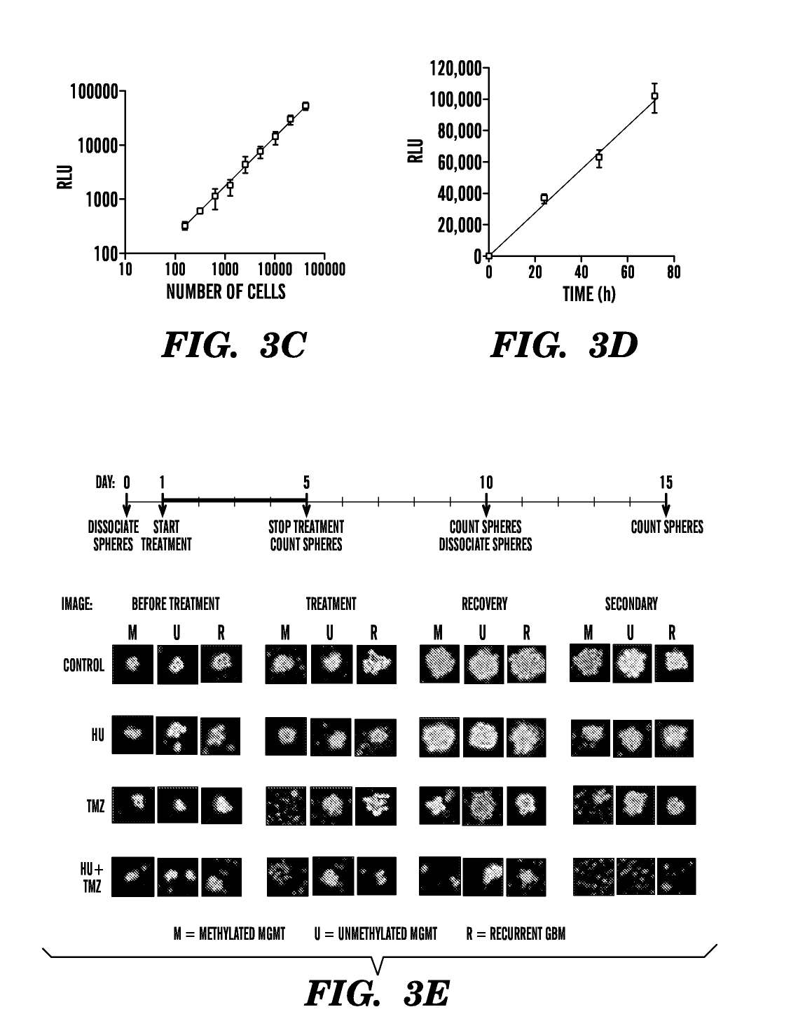

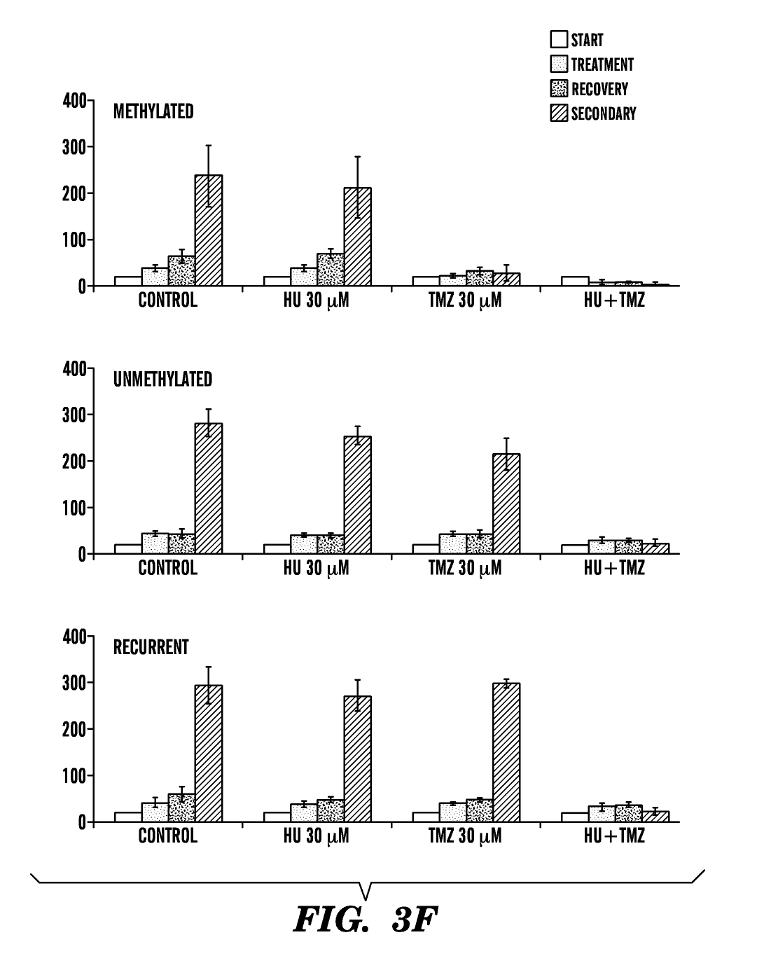

GBM cells with different MGMT promoter methylation status were obtained from newly diagnosed and recurrent patient tumor sections and grew as neural spheres in stem cells medium. Cells cultured this way retain the phenotype and genotype of primary tumors (Lee, J., et al., Cancer Cell 2006, 9, 391-403; Wakimoto, H., et al., Cancer research 2009, 69, 3472-3481) including MGMT promoter methylation status (Wakimoto, H., et al., Neuro Oncol 2012, 14, 132-144) and only cells with stem-like properties (e.g. Nestin/Sox2 expression) will form spheres and grow under these conditions (Wislet-Gendebien, S., et al., J Cell Sci 2003, 116, 3295-3302). GBM stem cells have been proposed to be the source of tumor recurrence and patient death (Chalmers, A.J.; DNA Repair (Amst) 2007, 6, 1391-1394), are resistant to conventional therapy (Bao, S., et al., Nature 2006, 444, 756-760), and can recapitulate a phenocopy of the original tumor upon implantation in nude mice (Singh, S. K., et al., Cancer research 2003, 63, 5821-5828). These cells were transduced with a lentivirus vector carrying the expression cassette for Gluc and the green fluorescent protein GFP at a multiplicity of infection of 10 transducing units per cells by adding the virus directly to the cells. This method of gene transfer yields >90% transduction efficiency in these cells as monitored by GFP expression (FIG. 3A). We confirmed expression of Nestin and CD133 (FIG. 3B). Since Gluc is naturally secreted, aliquots of conditioned medium can be assayed for its activity over time; thus, its secreted level correlated with cell viability (FIGS. 3C-3D) (Tannous, B. A., Nat Protoc 2009, 4, 582-591; Tannous, B. A., et al., Mol Ther 2005, 11, 435-443; Wurdinger, T., et al., Nat Methods 2008, 5, 171-173). The HU+TMZ (30 μM of each drug) combined therapy was applied on GBM neural spheres from newly diagnosed patients with methylated and unmethylated MGMT promoter as well as spheres from recurrent tumors. In culture, the combined HU+TMZ yielded an enhanced therapeutic effect on sphere formation, growth, and recovery, irrespective of their MGMT methylation status on both newly diagnosed and recurrent tumors (FIGS. 3E-3F). FACS analysis using AnnexinV/PI staining confirmed an increase in cell death upon combined therapy in all cells (data not shown).

### In Vivo Effect of HU on Primary GBM Cells In Vivo

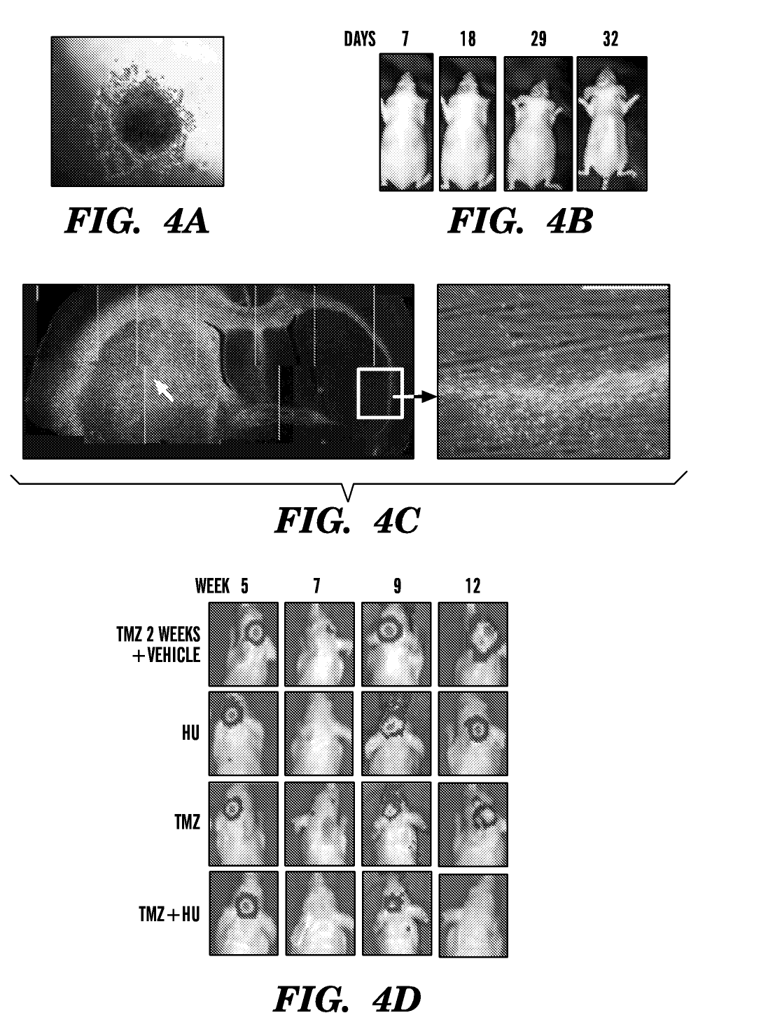

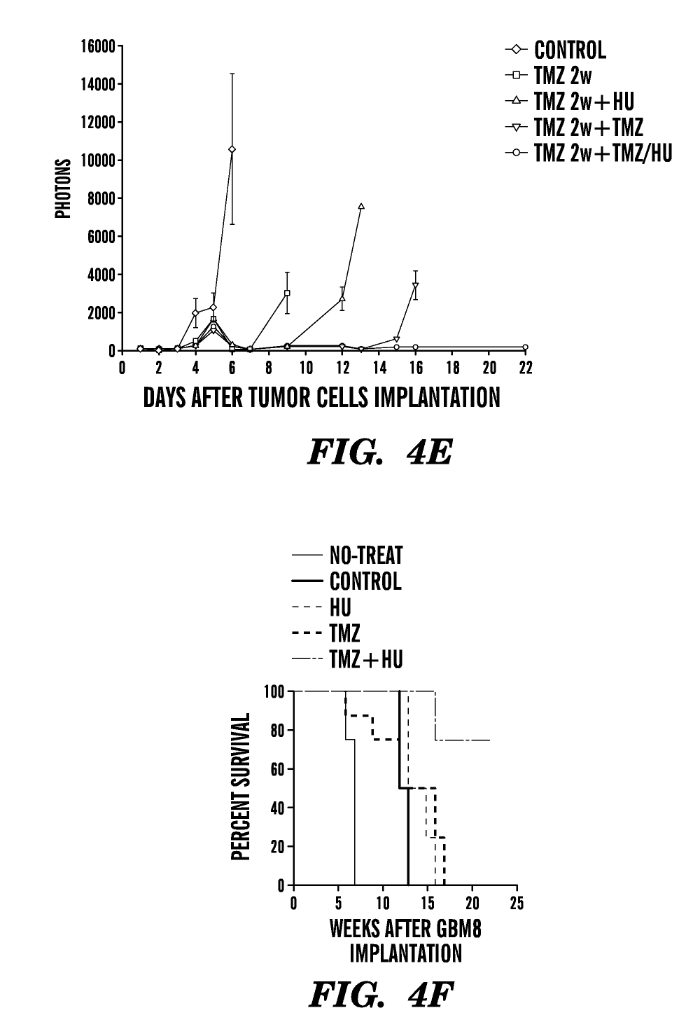

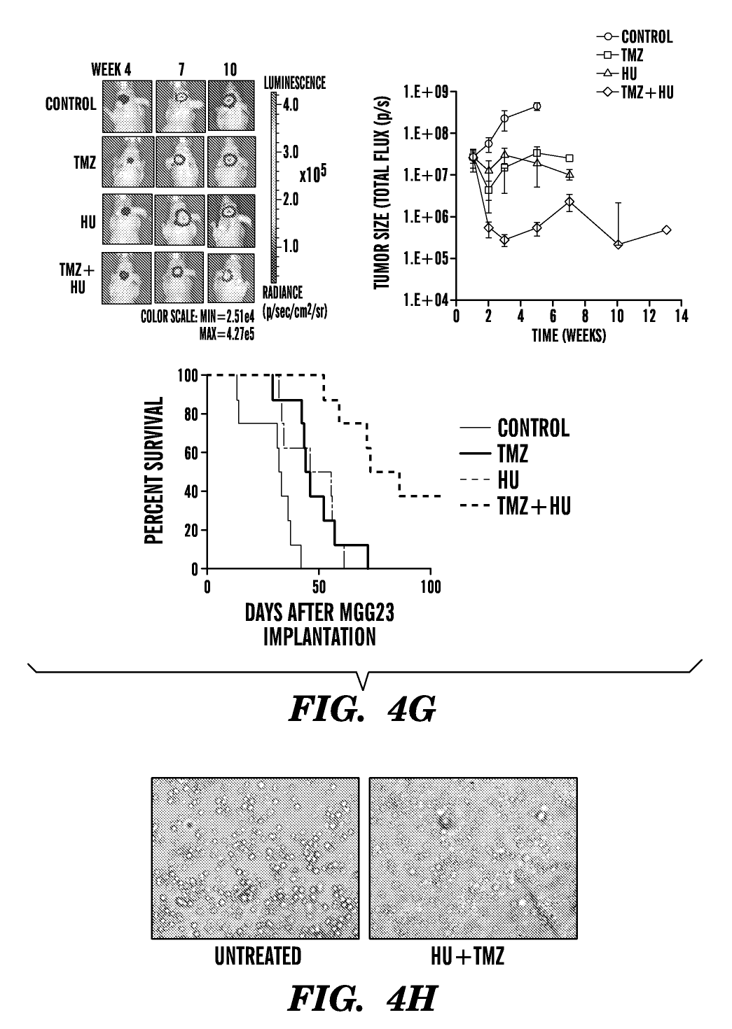

Primary GBM8 cells24 (from newly diagnosed tumor with methylated MGMT (Wakimoto, H., et al., Neuro Oncol 2012, 14, 132-144)) were grown in stem cell media as neural spheres. These cells were transduced with a lentivirus vector expressing Fluc and mCherry (Wurdinger, T., et al., Nat Methods 2008, 5, 171-173) at a multiplicity of infection of 10 yielding >90% infection efficiency. Upon intracranial injection, these cells formed tumors and infiltrated the brain of nude mice similar to human tumors (FIGS. 4A-4C). This model was used to test the efficacy of HU in combination with TMZ. Four weeks after implantation of 100,000 GBM8 cells, when tumors started to rapidly grow (as observed by Fluc imaging), mice were divided into two groups (n=20/group) which received DMSO vehicle or i.p. injection of 30 mg/kg TMZ four days/week over two weeks. All mice in the DMSO-treated group died by week seven after implantation of the GBM cells, whereas GBM8 tumors carrying methylated MGMT promoter responded very well to TMZ as expected (FIGS. 4D-4F). Mice were then left off TMZ to allow for GBM tumors to recur potentially becoming resistant to TMZ, recapitulating the patient scenario. At this point, the TMZ group was divided into four subgroups (n=10/group) which received either DMSO, HU (50 mg/kg), TMZ (30 mg/kg) or HU+TMZ four days/week over three weeks. The second round of TMZ treatment had a moderate effect on tumor volume but not a significant increase in survival rate. On the other hand, the combined HU+TMZ therapy had a remarkable and significant effect on tumor growth and survival rate with 80% of mice surviving over six weeks (compared to second round of TMZ alone) and remained tumor free (FIG. 4F). The same experiment was then performed on MGG23 GBM tumors carrying unmethylated MGMT promoter (Wakimoto, H., et al., Neuro Onco12012, 14, 132-144), and showed that these tumors also responded very well to the combined HU/TMZ therapy as observed by bioluminescence imaging and survival analysis (FIG. 4G). All together, these data supports the hypothesis that HU/TMZ combination could be used to treat newly diagnosed and recurrent GBM tumors irrespective of their MGMT status. Next, the toxicity of this combined therapy was assessed by treating mice with either DMSO, TMZ, HU, or HU+TMZ for four consecutive days and analyzing the blood for white blood cells (WBC), hematocrit (HCT), mean corpuscular volume (MCV), red cell distribution width (RDW), hemoglobin (HGB), mean corpuscular hemoglobin (MCH), red blood cells (RBC), platelets (PLT), and mean platelet volume (MPV). No significant difference between different treated groups in comparison to DMSO control was found (Table 1). Hematotoxicity on bone marrow was also evaluated by preparing bone marrow smears and counting viable cells from untreated and TMZ/HU-treated naïve mice as above. No apparent toxicity was observed with this combined therapy (FIG. 4H).

TABLE 1
Blood analysis to assess the toxicity of HU + TMZ therapy

| | Control | HU 30 mg/kg | TMZ 30 mg/kg | HU + TMZ |
|---|---|---|---|---|
| WBC | 4.8 ± 0.2 | 5.5 ± 1.0 | 4.4 ± 1.1 | 4.1 ± 1.6 |
| LYM | 3.9 ± 0.1 | 4.5 ± 0.9 | 3.2 ± 0.7 | 3.0 ± 1.1 |
| MONO | 0.2 ± 0.0 | 0.3 ± 0.1 | 0.3 ± 0.1 | 0.3 ± 0.1 |
| GRAN | 0.7 ± 0.1 | 0.7 ± 0.1 | 0.9 ± 0.4 | 0.8 ± 0.4 |
| LYM % | 81.1 ± 1.3 | 81.3 ± 1.3 | 75.5 ± 6.0 | 77.1 ± 2.7 |
| MONO % | 4.6 ± 0.4 | 4.2 ± 0.4 | 4.6 ± 1.1 | 4.3 ± 0.4 |
| GRAN % | 14.4 ± 1.3 | 14.5 ± 1.4 | 19.9 ± 5.0 | 18.6 ± 2.3 |
| HCT | 21.4 ± 1.4 | 23.7 ± 2.9 | 29.8 ± 1.4 | 17.4 ± 2.9 |
| MCV | 42.6 ± 0.6 | 42.4 ± 0.8 | 43.5 ± 0.7 | 42.0 ± 0.2 |
| RDWa | 31.4 ± 1.5 | 31.0 ± 1.6 | 32.8 ± 1.6 | 29.3 ± 0.5 |
| RDW % | 19.4 ± 1.0 | 19.4 ± 1.0 | 19.7 ± 0.7 | 18.1 ± 0.2 |
| HGB | 8.0 ± 0.5 | 8.9 ± 0.9 | 10.8 ± 0.5 | 6.7 ± 1.1 |
| MCHC | 37.6 ± 0.3 | 37.7 ± 0.9 | 36.3 ± 0.2 | 38.9 ± 0.4 |
| MCH | 16.0 ± 0.2 | 16.0 ± 0.2 | 15.8 ± 0.3 | 16.3 ± 0.2 |
| RBC | 5.0 ± 0.3 | 5.6 ± 0.6 | 6.9 ± 0.2 | 4.1 ± 0.7 |
| PLT | 184.5 ± 38.2 | 234.7 ± 28.8 | 122.0 ± 25.7 | 162.7 ± 21.6 |
| MPV | 6.2 ± 0.2 | 6.3 ± 0.4 | 6.5 ± 0.4 | 6.2 ± 0.2 |

### Mechanism of Action of HU

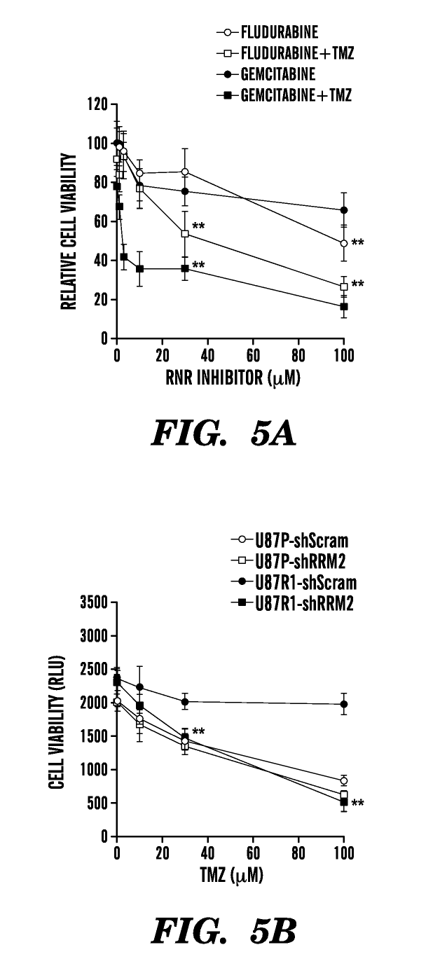

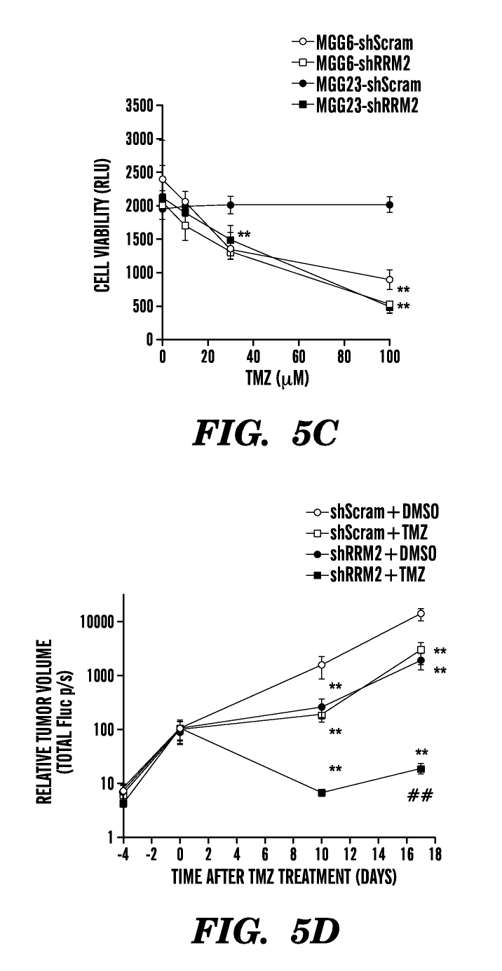

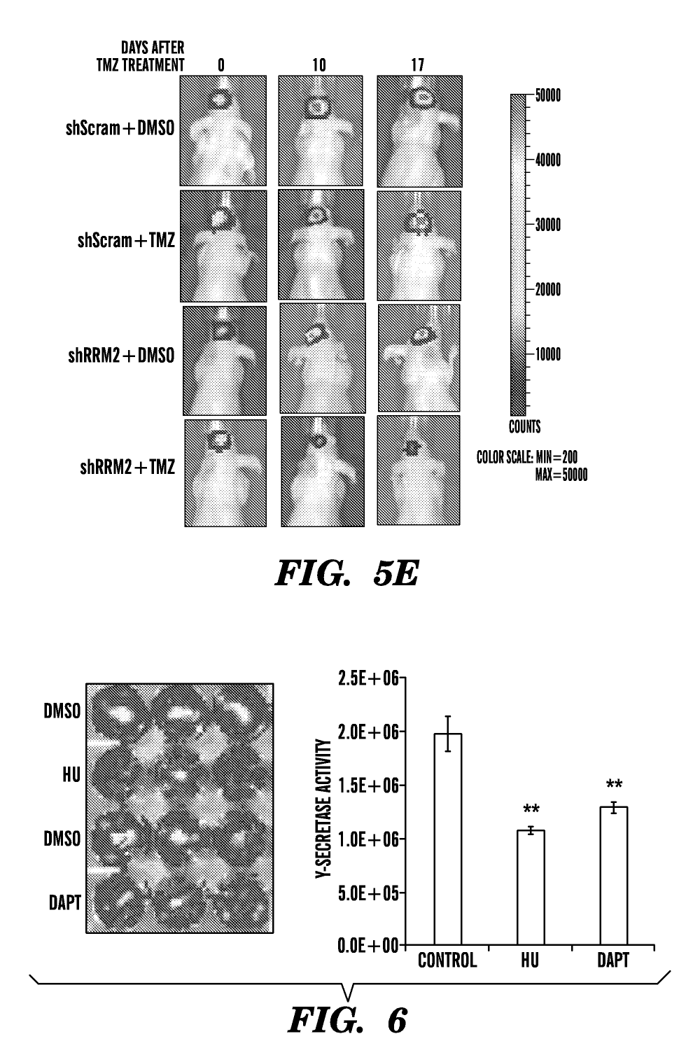

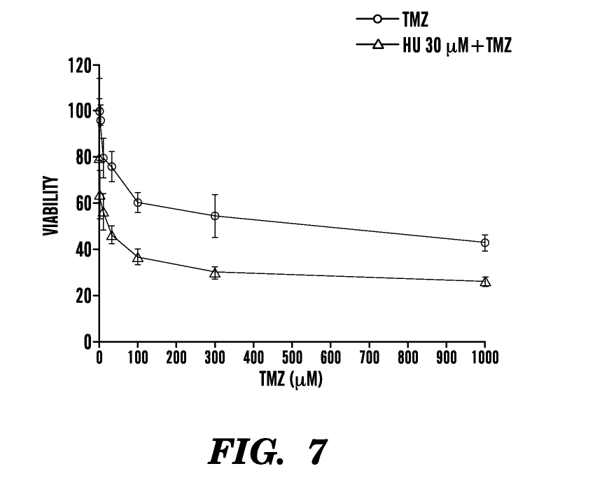

Since HU is known to target ribonucleotide reductase subunit 2 (RNR), knockdown experiment was first performed in different cell lines using shRNA against RNR (shRNR) or scrambled control (ShScram) and different concentrations of TMZ. As expected, RNR knockdown sensitized all glioma cells inclusing U87P, U98R, MGG6 (methylated MGMT promoter) and MGG23 (Unmethylated MGMT) to TMZ irrespective of MGMT status (FIGS. 5B-5C). Further, the same sensitization effect was observed on U87 glioma cells implanted intracranially and expressing shRNR (FIG. 5D). Other ribonucleotide reductase (RNR) inhibitors were further evaluated to determine if a similar effect as HU is obtained on GBM. GBM cells from recurrent tumors expressing Gluc were treated in a 96-well plate with either fludarabine or gemcitabine—classical RNR inhibitors—and cell viability was assessed by the Gluc assay. As expected, TMZ alone had a moderate-to-no effect on these cells. Both RNR inhibitors sensitized GBM cells to TMZ suggesting that RNR is a key player in the synergistic effect (FIG. 5A). The effect of HU on Notch signalling was also explored since this pathway had been suggested as a target for cancer therapy (Ranganathan, P., et al., Nat Rev Cancer 2011, 11, 338-351), and Notch inhibitors have been reported to synergize with TMZ in GBM (Chu, Q., et al., Clinical cancer research: an official journal of the American Association for Cancer Research 2013, 19, 3224-3233; Gilbert, C. A., et al., Cancer research 2010, 70, 6870-6879; Ulasov, I. V., et al., Molecular medicine 2011, 17, 103-112). The RBP-Jk-Fluc reporter plasmid which carries RBP-Jk transcriptional response element driving the expression of Fluc34 was used. RBP-Jk is a DNA binding transcription factor and a direct downstream modulator of Notch signalling. Thus, this reporter can indirectly monitor Notch activity (Minoguchi, S., et al., Mol Cell Biol 1997, 17, 2679-2687). 293T cells were transfected with this plasmid, treated with 30 μM HU, and monitored Fluc activity 3 days later. Interestingly, HU yielded 50% decrease in Fluc activity and therefore inhibited Notch signalling (FIG. 6). Notch signalling is activated through an interaction of Notch receptor with a ligand expressed on adjacent cells leading to proteolytic cleavages of Notch receptor, catalyzed by the γ-secretase complex (Grosveld, G. C,. Nat Med 2009, 15, 20-21) Inhibition of γ-secretase by DAPT resulted in a decrease of Fluc activity similar to HU (FIG. 6). These data suggest that HU could target Notch pathway and acts as an RNR inhibitor, and therefore could have a “dual” therapeutic effect on GBM tumors.

Hydroxyurea (HU) was used as adjuvant therapy for glioblastoma since HU was found to sensitize GBM cells to TMZ, irrespective of their MGMT promoter methylation status. HU is a simple organic compound that acts specifically on the S-phase of the cell cycle by inhibiting the enzyme ribonucleotide reductase, thereby hindering the reductive conversion of ribonucleotides to deoxyribonucleotides and thus limiting de novo DNA synthesis (Koc, A., et al., J Biol Chem 2004, 279, 223-230). This property makes HU an attractive candidate for cancer therapy. One advantage for the use of HU for brain tumors is that it increases the blood brain tumor permeability of certain chemotherapeutics and therefore should enhance penetration of TMZ to GBM (Yin, D., et al. Clinical cancer research: an official journal of the American Association for Cancer Research 2008, 14, 4002-4009). Since HU is FDA-approved and have been used to treat myeloproliferative diseases (Boyd, A. S. & Neldner, K. H., Journal of the American Academy of Dermatology 1991, 25, 518-524; Streiff, M. B., et al., Blood 2002, 99, 1144-1149), sickle cell anaemia (Charache, S., et al., The New England journal of medicine 1995, 332, 1317-1322) as well as some forms of tumors (such as melanoma, ovarian, squamous cell carcinoma, head and neck carcinoma and brain tumors) (Madaan, K., et al., Expert review of anticancer therapy 2012, 12, 19-29) in combination with other anti-cancer agents (never with TMZ), it is relatively easy translated to the clinic.

This is the first demonstration for the use of HU as adjuvant therapy for GBM in combination with TMZ. HU was evaluated for the treatment of both newly diagnosed GBM as well as recurrent, TMZ-resistant, tumors. Cells obtained from patient tumor tissues were employed (with different MGMT status and genetic modifications), which infiltrate the brain of mice similar to human tumors upon intracranial injection. HU synergizes with TMZ, on both newly diagnosed and recurrent tumors, irrespective of the MGMT promoter methylation status. Although HU has been previously evaluated in malignant gliomas in combination with radiation or cytotoxic chemotherapy and has shown limited efficacy (Prados, M. D., et al., Int J Radiat Oncol Biol Phys 1998, 40, 57-63; Kyritsis, A. P., et al., Neurosurgery 1996, 39, 921-926; Levin, V. A. & Prados, M. D., J Clin Oncol 1992, 10, 766-771), it was not evaluated with an agent with validated efficacy in GBM such as TMZ.

### Lentivirus Vectors

The Gluc cDNA (Tannous, B. A., et al., Mol Ther 2005, 11, 435-443), and the GFP expression cassette separated by an internal ribosomal entry site (IRES) element has been cloned into a lentivirus vector under the control of the strong constitutive cytomegalovirus (CMV) promoter. Similar vector has been generated to express Fluc and RFP. Lentivirus vector stocks are produced as previously described (Wurdinger, T., et al., Nat Methods 2008, 5, 171-173). Vectors are titered based on fluorescent protein expression as transducing units (tu) with titers usually around 10⁸ tu/ml. Different cell types will be infected with these lentivirus vector at a multiplicity of infection of 10 by adding the vector directly to the cells which give >90% transduction efficiency in GBM cells and spheres (Tannous, B. A., Nat Protoc 2009, 4, 582-591; Wurdinger, T., et al., Nat Methods 2008, 5, 171-173).

### GBM Stem-Like Cells

GBM cells with different MGMT promoter methylation status were obtained from newly diagnosed and recurrent patient tumor sections and grew as neural spheres in stem cell medium [neurobasal medium supplemented with EGF and FGF (20 ng/mL), heparin (1:1000), B27 supplement (1:50), LIF (1:1000)].

### Cell Cycle Analysis

After treatment, cells are incubated with 10 μM BrdU-FITC (BD Biosciences) for 45 min (time will be optimized in case of problems), washed with PBS, and fixed in 70% ethanol. Prior to analysis by flow cytometry, cells are washed and incubated with 0.15 mg/ml RNAse A for 20 min followed by 50 μg/μl of propidium iodide (PI) for 30 min at 37° C. Cell cycle phase distribution (G₀/G₁, S, G₂/M phases) assessment is performed using CellQuest software as previously described (Mir, S. E., et al., Cancer Cell 2010, 18, 244-257).

### Normal Stem Cells

Normal human mesenchymal stem cells from normal bone marrow (Lonza, cat # PT-2501) or from human adipose tissues (Invitrogen, R7788-110) were cultured as per manufacture instructions. Both of these normal stem cells were be differentiated into the three neural lineages using the differentiation kit (Stem Cell Technologies) (Strem, B. M., et al., Keio J Med 2005, 54, 132-141). These cells were stained for Tubulin beta III (neuron specific), Glial fibrillary acidic protein (astrocytes) and anti-oligodendrocyte clone NS-1 antibody (oligodendrocytes) to confirm differentiation.

### In Vivo Models

GBM cells were first transduced with a lentivirus vector to stably express Fluc. Fifty thousands of these cells were intracranially injected in the brain of nude mice using the following coordinates from the bregma in mm: anterior-posterior +0.5 mm, medio-lateral +2.0 mm, dorso-ventral −2.5 mm. One week after implantation of 50,000 (low amount) or 200,000 (high amount) GBM cells, or 100 GBM spheres, mice were randomized into different treatment groups. Tumor growth was monitored over time by in vivo Fluc bioluminescence imaging after i.p. injection of 200 mg/kg D-luciferin substrate and acquiring signal using a cooled CCD camera (Wurdinger, T., et al., Nat Methods 2008, 5, 171-173).

1. A method of treating a tumor in a subject, the method comprising administering to the subject a therapeutically effective amount of a DNA damaging agent and a ribonucleotide reductase inhibitor.

2. The method of claim 1, wherein the ribonucleotide reductase inhibitor is selected from a group consisting of hydroxyurea, motexafin gadolinium, fludarabine, cladribine, gemcitabine, tezacitabine, triapine, gallium maltolate, gallium nitrate, and a combination thereof.

3. The method of claim 1, wherein the ribonucleotide reductase inhibitor is hydroxyurea, fludarabine, gemcitabine, or a combination thereof.

4. The method of claim 1, wherein the DNA damaging agent is not a ribonucleotide reductase inhibitor.

5. The method of claim 1, wherein the DNA damaging agent is not radiation.

6. The method of any of claims 1 5 claim 1, wherein the DNA damaging agent is a chemotherapeutic agent or a PARP inhibitor.

7. The method of claim 6, wherein the chemotherapeutic agent is selected from a group consisting of temozolomide (TMZ), bendamustine, irinotecan, capecitabine, topotecan, cisplatin, oxaliplatin, carboplatin, nedaplatin, satraplatin, triplatin tetranitrate, camptothecin, cytarabine, fluorouracil, cyclophosphamide, etoposide phosphate, teniposide, doxorubicin, daunoaibicin, pemetrexed, mitomycin C, chlorambucil, and melphalan.

8. The method of claim 7, wherein the chemotherapeutic agent is temozolomide (TMZ).

9. The method of any of claims 1 g claim 1, wherein the ratio of the DNA damaging agent to the ribonucleotide reductase inhibitor is sufficient to sensitize tumor cells to the DNA damaging agent.

10. The method of claim 1, wherein the tumor is selected from a group consisting of central nervous system (CNS) neoplasm, melanoma, recurrent adult acute lymphoblastic leukemia, recurrent childhood acute lymphoblastic leukemia, Ewing sarcoma, unspecified adult solid tumor, unspecified childhood solid tumor, hepatocellular carcinoma, pancreatic neuroendocrine tumor (e.g., gastrinoma, glucagonoma, insulinoma, islet cell carcinoma, pancreatic polypeptide tumor, recurrent islet cell carcinoma, or somatostatinoma), lung cancer, colorectal cancer, rectal cancer, breast cancer, ovarian cancer, rhabdomyosarcoma, acute myelogenous leukemia, and myelodysplastic syndrome.

11. The method of claim 10, wherein the CNS neoplasm is selected from a group consisting of glioma, glioblastoma, oligodendroglioma, astrocytoma, medulloblastoma, oligoastrocytoma, gliosarcoma, recurrent adult brain tumor, B-cell lymphoma originating in the CNS, childhood high-grade cerebellar astrocytoma, childhood high-grade cerebral astrocytoma, childhood spinal cord neoplasm, childhood brain stem glioma, childhood cerebral astrocytoma, peripheral primitive neuroectodermal tumor, recurrent childhood medulloblastoma, recurrent childhood supratentorial primitive neuroectodermal tumor, and recurrent childhood pineoblastoma.

12. The method of claim 1, wherein the tumor is glioblastoma or melanoma.

13. The method of claim 12, wherein the glioblastoma is recurrent glioblastoma.

14. The method of claim 1, wherein the tumor is resistant to the DNA damaging agent.

15. The method of claim 1, wherein the ribonucleotide reductase inhibitor is administered before the administration of the DNA damaging agent.

16. The method of claim 1, wherein the ribonucleotide reductase inhibitor is administered simultaneously with the administration of the DNA damaging agent.

17. The method of claim 1, wherein the ribonucleotide reductase inhibitor is administered after the administration of the DNA damaging agent.

18. (canceled)

19. (canceled)

20. The method of claim 1, wherein the subject is a mammal.

21. The method of claim 20, wherein the subject is a human.

22-26. (canceled)

27. A pharmaceutical composition comprising an effective amount of a DNA damaging agent and a ribonucleotide reductase inhibitor.

28-34. (canceled)
